(19) United States
(12) Patent Application Publication
(10) Pub. No.: US 2024/0390595 A1
(43) Pub. Date: Nov. 28, 2024

(54) DOSING OPERATION SPEED DETECTION WITH ELECTRONIC SYSTEM FOR A DRUG DELIVERY DEVICE

(71) Applicant: Sanofi, PARIS (FR)

(72) Inventors: Ronald Smith, Salir, Loule (PT); Paul Richard Draper, Warwick (GB); John Whitehouse, Harvington, Evesham (GB); Hester Jane Corne, Warwick (GB)

(21) Appl. No.: 18/694,093

(22) PCT Filed: Sep. 22, 2022
(86) PCT No.: PCT/EP2022/076283

§ 371 Date: Mar. 21, 2024

(30) Foreign Application Priority Data
Sep. 24, 2021 (EP) 21315170.7

(51) Int. Cl. A61M 5/315

(52) U.S. Cl. CPC A61M 5/31568; A61M 2205/33; A61M 2205/3317

(57) ABSTRACT

The present invention relates to an electronic system (100) for a drug delivery device (1), in which a first member (20) performs a specific movement relative to a second member (11) during a dosing operation. The electronic system (100) comprises a sensor arrangement (120), which is operable to provide sensor data describing the specific movement and is configured to operate the sensor arrangement (120) during at least one part of the dosing operation to provide sensor data. In order to protect the users from relying on incorrectly measured dose sizes and a need to reduce the risk that an injection is painful for the patient, the electronic system (100) is configured to determine, based on the sensor data, if a speed of the specific movement during the at least one part of the dosing operation breaches a first speed threshold. Additionally or alternatively, the electronic system (100) is configured to determine, based on the sensor data, the speed of the specific movement and/or the dosing operation during the at least one part of the dosing operation.

The present invention relates to an electronic system for a drug delivery device, in which a first member performs a specific movement relative to a second member during a dosing operation, wherein the electronic system comprises a sensor arrangement, which is operable to provide sensor data describing the specific movement, and wherein the electronic system is configured to operate the sensor arrangement during at least one part of the dosing operation to provide sensor data. The present invention further relates to a drug delivery device comprising the electronic system and a method for operating the electronic system.

Pen type drug delivery devices have application where regular injection by persons without formal medical training occurs. This may be increasingly common among patients having diabetes where self-treatment enables such patients to conduct effective management of their disease. In practice, such a drug delivery device allows a user to individually select and dispense a number of user variable doses of a medicament.

There are basically two types of drug delivery devices: resettable devices (i.e., reusable) and non-resettable (i.e., disposable). For example, disposable pen delivery devices are supplied as self-contained devices. Such self-contained devices do not have removable pre-filled cartridges. Rather, the pre-filled cartridges may not be removed and replaced from these devices without destroying the device itself. Consequently, such disposable devices need not have a resettable dose setting mechanism. The present invention is applicable for disposable and reusable devices.

For such devices the functionality of recording doses that are dialed and delivered from the pen may be of value to a wide variety of device users as a memory aid or to support detailed logging of dose history. Thus, drug delivery devices using electronics are becoming increasingly popular in the pharmaceutical industry as well as for users or patients. For example, a drug delivery device is known from EP 2 729 202 B1 comprising an electronically controlled capturing system for capturing data related to the amount of drug expelled from a reservoir by expelling means.

Especially if the device is designed to be self-contained, that is to say without a connector for a connection to an electric power source which is necessary to provide electric power for the operation of the device, the management of the resources of a power supply integrated into the device is particularly important. Unpublished patent applications EP 20315066.9 and EP 20315357.2 disclose embodiments of electronic systems for drug delivery devices with improved power management. Unpublished patent application EP 20315451.3 discloses an electronic system for a drug delivery device comprising a use detection unit and a drug delivery device comprising the electronic system. The electronic system comprises a rotationally actuated mechanical switch (rotary switch). The rotary switch indicates that a dose delivery operation has begun.

Unpublished patent specification EP 20315305.1 discloses a further electronic control system for a drug delivery device. The electronic control system comprises an electrical motion sensing unit that can be used to determine the size of doses delivered by the drug delivery device. The size of the dose is determined based on a movement of a first member relative to a second member of a dose setting and drive mechanism of the drug delivery.

It may occur that a speed of the first member relative to the second member during a dose delivery operation and/or during a dose setting is very fast. In particular, the speed might be so high that a maximum time resolution of the motion sensing unit is exceeded.

Consequently, the motion sensing unit is no longer able to provide measurement data that ensures a correct and accurate determination of the size of the dose. Typically, the determined size of the dose will be less than the size of the dose that was actually delivered.

Apart from this, a fast dispense speed (a speed of dose delivery operation) increases the risk that the injection is painful for the patient.

Therefore, there is a need to protect the users from relying on incorrectly measured dose sizes and a need to reduce the risk that an injection is painful for the patient.

This object is solved by an electronic system for a drug delivery device according to claim 1.

It should be noted that the disclosure is not restricted to the subject matter defined in the appended claims. Rather, the disclosure may comprise improvements in addition or as an alternative to the ones defined in the independent claims as will become apparent from the following description.

In the drug delivery device, a first member performs a specific movement relative to a second member during a dosing operation.

The electronic system comprises a sensor arrangement, which is operable to provide sensor data describing the specific movement. Further, the electronic system is configured to operate the sensor arrangement during (at least one part of) the dosing operation to provide sensor data.

The electronic system is configured to determine, based on the sensor data, if a speed of the specific movement during (the at least one part of) the dosing operation breaches a first speed threshold. Additionally or alternatively, the electronic system is configured to determine, based on the sensor data, the speed of the specific movement and/or the dosing operation during (the at least one part of) the dosing operation.

The present invention ensures automatic detection of unfavourably high and/or low speeds.

Especially, the electronic system may be configured to determine, based on the sensor data, if the speed of the specific movement during (the at least one part of) the dosing operation exceeds the first speed threshold. In this case, the first speed threshold is an upper threshold. This helps to support the training of the injection behaviours of patients, health care professionals (HCP), and other users in order to avoid excessive speeds during dosing operation. The first speed threshold can be indicative of an increased risk that the injection is painful for the patient. Additionally or alternatively, the first threshold can be indicative that a maximum time resolution of the sensor arrangement is almost reached, reached, or exceeded. In other words, the electronic system is able to detect the approach of a measurement limit of the sensor arrangement. Therefore, the present invention helps to protect the users from relying on incorrectly measured dose sizes and to reduce the risk that an injection is painful for the patient.

Additionally or alternatively, the electronic system may be configured to determine, based on the sensor data, if the speed of the specific movement during (the at least one part of) the dosing operation falls below the first speed threshold. In this case, the first speed threshold is a lower threshold.

In one embodiment, the electronic system is configured to determine, based on the sensor data, if the speed of the specific movement during (the at least one part of) the dosing operation breaches the first speed threshold without determining the actual value of the speed in general. For example, the actual value of the speed might be not important in case the actual value is well above or well below the first speed threshold.

Preferably, the electronic system comprises an electronic control unit configured to control operation of the electronic system. The sensor arrangement may be operatively connected to the electronic control unit and the electronic control unit may be configured to operate the sensor arrangement during (the at least one part of) the dosing operation to provide the sensor data. Furthermore, the electronic control unit may be configured to determine, based on the sensor data, if the speed of the specific movement during (the at least one part of) the dosing operation breaches the first speed threshold.

By providing the sensor arrangement separately from the electronic control unit, the production costs can be reduced. Furthermore, the sensor arrangement as such can be made smaller and can be better adapted to the construction and/or shape of the drug delivery device and its components. This facilitates implementation of the electronic system within the drug delivery device.

As noted above, the first member performs the specific movement relative to the second member during dosing operation. Vice versa, (progress of) the dosing operation may require the occurrence of the specific movement. Preferably, the specific movement occurs only during dosing operation.

Preferably, the electronic system comprises an electrical power supply. The electrical power supply may comprise, for example, a rechargeable battery, a non-rechargeable battery, a solar cell, and/or an inductive power supply. The electrical power supply may be electrically connected at least with the electronic control unit.

In one embodiment, the electronic system comprises a memory. Especially, the electronic control unit may comprise the memory or the memory may be operatively connected to the electronic control unit. In one embodiment, (at least a part of) the memory is non-volatile. Thus, even if the power supply to the electronic system is reduced and/or if the electronic system is switched off, information stored in the memory may still be available for subsequent operations.

According to another aspect, the electronic system may comprise a communication unit. Especially, the electronic control unit may comprise the communication unit or the communication unit may be operatively connected to the electronic control unit. The communication unit may be configured for the transmission of data (from the electronic system, especially from the electronic control unit) to a second device. Additionally or alternatively, the communication unit may be configured to receive data from the second device.

For example, the second device includes or consists of a mobile phone, a tablet, a personal computer and/or another medical device, such as a blood glucose meter.

According to a further aspect of the present invention, the communication unit is not operated (shut off) in a sleep state and/or in a measurement state. This reduces the electrical power consumption of the electronic system. Preferably, the communication unit is only operated (switched on) in a synchronization state (explained below) and/or a pairing state.

The pairing state may be for establishing data connection to the second device for allowing the transfer of data from the electronic system to the second device and/or from the second device to the electronic system.

The communication may be configured for wired transfer of data and/or for wireless transfer of data.

In one embodiment, the communication unit comprises a wireless communications interface for communicating with the second device via a wireless network such as Wi-Fi or Bluetooth®, and/or an interface for a wired communications link, such as a socket for receiving a Universal Serial Bus (USB), mini-USB or micro-USB connector. For example, the communication unit may comprise a Bluetooth® core. The Bluetooth® core may be a non-programmable, fixed processing core. It may be configured to handle all low-level Bluetooth® communications functionality to prove the Bluetooth® interface for a main microcontroller of the electronic system to use.

In one embodiment, the electronic system is configured to provide a first speed warning depending on whether the specific movement during (the at least one part of) the dosing operation breaches the first speed threshold (i.e. exceeds the first speed threshold being an upper threshold or falls below the first speed threshold being a lower threshold). Providing the first speed warning may include
generating a visual, audible, and/or tangible alert (for a user), and/or
transmitting a first speed warning signal by means of the communication unit, for example to the second device.

The electronic system (especially the electronic control unit) is preferably configured to generate a data record including a first speed indication for the dosing operation depending on whether the specific movement during (the at least one part of) the respective dosing operation breaches the first speed threshold. By this, the first speed indication for the respective dosing operation can be provided and/or stored. For example, the first speed indication may include or consist of a corresponding flag and/or a corresponding specific purpose value.

In more detail, the electronic system may be configured to generate the data record including the first speed indication for the dosing operation if the specific movement during (the at least one part of) the respective dosing operation breaches the first speed threshold. According to a further aspect, no first speed indication or even no data record may be provided else.

In one embodiment, the electronic system may be configured to generate the data record including the first speed indication for the dosing operation if the specific movement during (the at least one part of) the respective dosing operation does not breach the first speed threshold. According to a further aspect, no first speed indication or even no data record may be provided else.

In one embodiment, the electronic system is configured to generate, for the respective dosing operation, a data record including a certain data field, wherein the certain data field is set a first value (as the first speed indication) if the specific movement during (the at least one part of) the respective dosing operation breaches the first speed threshold and to a different value else. The different value can be a pre-determined second-value. According to another aspect, the different value can be a variable value different from the first value, for example a size of the dose and/or a time stamp for the respective dosing operation.

In a preferred embodiment, a corresponding flag is raised in the data record in case the specific movement during (the at least one part of) the respective dosing operation breaches the first speed threshold. In more detail, the data record may include a corresponding flag field, wherein said flag field is set to a (first) value corresponding to ‘true’ in this case but set to a (second) value corresponding to ‘false’ else.

In one embodiment, the electronic system (especially the electronic control unit) is configured to transmit the data record to the second device, for example by using the communication unit. This allows the electronic system (especially the electronic control unit) to provide the data record to the second device. This can be used to create a back-up of the data record. Furthermore, the second device may be configured to evaluate the data records. The second device may allow the HCP, the manufacturer of the drug delivery device, and/or another user (for example parents of a child using the drug delivery device) to receive, access, view, and/or evaluate the data.

According to one aspect, the electronic system is configured to store the data record in the memory, especially in the non-volatile memory. By this, the data record is made available for later evaluation, for example by the electronic system itself, by the user, the HCP, the manufacturer, and/or the second device. For example, this allows transmitting the data record later to the second device if the second device is not paired and/or connected to the electronic system at the time of the respective dosing operation.

In particular, the electronic system may be configured to store a plurality of data records in the memory, for example at least the ten latest data records, more preferably at least the latest fifty data records.

In a preferred embodiment, the electronic system (especially the electronic control unit) is configured to operate the sensor arrangement periodically with at least a first sample rate. This may apply at least during (the at least one part of) the dosing operation and/or in the measurement state. This ensures a pre-defined sensing interval (time) between subsequent individual sensor readings. The sensor data for the dosing operation may include a plurality of individual sensor readings. If the sample rate for subsequent individual sensor readings is known, the corresponding sensing interval can be considered for estimating the speed of the specific movement between different individual sensor readings, for example between two consecutive individual sensor readings.

According to a further aspect, the electronic system (especially the electronic control unit) is configured to operate the sensor arrangement at least with a first sample rate and a second sample rate, wherein the first sample rate is higher than the second sample rate. In other words, the electronic system (especially the electronic control unit) is configured to operate the sensor arrangement with at least two different sample rates. This may apply at least during (the at least one part of) the dosing operation and/or in the measurement state. With the first sample rate, more individual sensor readings per time unit are generated and provided. A (first) sensing time interval is shorter. The sensor data can describe the specific movement more precisely, especially if the speed is high. Accordingly, the speed can be checked, estimated, and/or determined more precisely and reliably. On the other hand, operating the sensor arrangement with the second sample rate reduces the electrical power consumption. This helps to save electrical power and to increase a lifetime of the electrical power supply. In one embodiment, the first sample rate may be also denoted as “fast sample rate” and the second sample rate may be also denoted as “base sample rate”.

Using different sample rates allows the electronic system (especially the electronic control unit) to adapt the use of the sensor arrangement to different conditions and measurement requirements while optimizing the energy efficiency. An individual sample rate may be chosen to offer power consumption benefits. For example, the second sample rate may be chosen when no high measurement accuracy is required. An individual sample rate may be selected to ensure that as much sensor data as possible is captured. For example, the first sample rate may be a maximum sample rate. If the speed of dosing operation is higher than corresponding to the maximum sample rate, transitions (indicating changes between different positions of the first member relative to the second member that can be distinguished by the sensor arrangement) may occur in between consecutive sensor readings. Such transmissions may be hence “missed”: They will not be detected by the sensor arrangement.

The different sample rates may be determined by the electronic system itself, in particular by its hardware. For example, the first sample rate may be determined by hardware limitations of the sensor arrangement and/or a sensor controller of the electronic control unit.

According to another aspect, at least one of, several of, or all of the different sample rates—for example at least the second sample rate—may by pre-programmed.

In one embodiment, the first sample rate is pre-set (for example pre-programmed) to a particular value. In more detail, the first sample rate may be pre-set based on an expected maximum speed of the dosing operation. For example, the expected maximum speed may be a dispense speed above which the user is extremely unlikely to dispense. Additionally or alternatively, the drug delivery device may not physically allow dosing operation faster than the expected maximum speed (preferably at least not during normal operation), e.g. due to limitations in a dose setting and drive mechanism of the drug delivery device. The expected maximum speed may be determined by laboratory testing and/or simulations. The first sample rate may be pre-set to the expected maximum speed, to the expected maximum speed times a certain factor (for example 1.2 in order to ensure that the first sample rate is sufficiently high in any expected case), or the expected maximum speed plus a certain value. The first sample rate may be set sufficiently high that a very fast speed threshold explained can be defined between the expected maximum speed and the first sample rate.

The expected maximum speed may be the same or different for a dispense speed (speed during dose delivery operation) and a setting speed (speed during dose setting operation). Accordingly, if (the at least one part of) the dosing operation includes both (at least one part of) the dose setting operation and (at least one part of) the dose delivery operation, different maximum sample rates may be pre-set for (the at least one part of) the dose setting operation and (the at least one part of) the dose delivery operation. It is also possible to pre-set a common maximum sample rate based on the larger one of the expected maximum setting speed and the expected maximum dispense speed.

With the first sample rate, fast dosing operations can be accurately detected, described, and/or measured. For example, particularly fast speed between the first member and the second member can occur if a dose delivery operation is performed while no cartridge is fitted (because there is no resistance against advancement of a piston rod by a bung of the container) or if a patient is dispensing into air (because there is less resistance of expelling the medicament out of a needle).

The individual sample rates can be fractions of 1 Hz, up to many GHz, or higher. The sample rate(s) may depend on the technology employed in the electronic system. The first sample rate may be at least 1000 Hz, more preferably at least 2000 Hz, for example at least 3500 Hz. Additionally or alternatively, the first sample rate may be less than 15000 Hz, more preferably less than 6000 Hz. The second sample rate may be at least 100 Hz, more preferably at least 300 Hz, most preferably at least 450 Hz. Additionally or alternatively, the first sample rate may be less than 1000 Hz, more preferably less than 600 Hz.

In one embodiment, the electronic system (especially the electronic control unit) is configured to operate the sensor arrangement with exactly two different sample rates. In another embodiment, the electronic system (especially the electronic control unit) is configured to operate the sensor arrangement with at least three different sample rates.

In one embodiment, the electronic system, preferably specifically the electronic control unit, is configured to operate the sensor arrangement with the second sample rate and to increase the sample rate to the first sample rate when the sensor data indicates occurrence of the specific movement. This may apply at least during (the at least one part of) the dosing operation and/or in the measurement state. In this regard, the electronic system detects the occurrence of the specific movement based on the sensor data. For example, if the sensor data comprises or consist of Gray code data, the electronic system (e.g. the electronic control unit) may be configured to operate the sensor arrangement with the first sample rate upon any transition, especially upon any forward transition, for example upon detecting that the Gray code has incremented. Naturally, a change of the sensor data can be detected also while the sensor arrangement is operated with a sample rate that is smaller than the first sample rate, for example with the second sample rate. The indication of the occurrence of the specific movement shows that the relevant dosing operation is in progress. In order to provide reliable and precise sensor data, particularly also in cases with high speeds (for example if dose delivery operation is performed while no cartridge is fitted or if the user dispenses into air), the electronic system applies the first sample rate immediately upon the indication.

According to a further aspect, the electronic system (especially the electronic control unit) may be configured to reduce the sample rate below the first sample rate, for example to the second sampling rate,
after having operated the sensor arrangement continuously with the first sample rate for a predetermined time and/or
having obtained a pre-defined number of sensor readings in one turn with the first sample rate.

This may apply at least during (the at least one part of) the dosing operation and/or in the measurement state. The reduction of the sample rate helps to save electrical power, in particular even in the measurement state. In other embodiments, alternative configurations using one or several sample rates in a different way can be implemented.

The pre-defined number of sensor readings is preferably a number in the range from 5 to 100, more preferably in the range from 8 to 40, most preferably in the range from 10 to 20. In other words, the sample rate is automatically reduced under pre-defined circumstances.

The certain limit may be a pre-defined value. In more detail, the certain limit may be based on a time resolution of the sensor arrangement while a sample rate smaller than the first sample rate (for example the second sample rate) is applied. This prevents, the reduced sample rate being used while the specific movement progresses with a high speed exceeding the measurement accuracy of the reduced sample rate.

Counting of the pre-defined period of time and/or the pre-defined number of sensor readings may be reset when the sensor data indicates any new (or further) occurrence of the specific movement, for example if there are any new forward transitions. This prevents the sample rate being reduced when transitions continue to occur.

Preferably, the electronic system comprises a switch configured to provide a use signal upon
transition to dose delivery operation, and/or
the occurrence of the specific movement.

The switch may be operatively connected to the electronic control unit. The switch may comprise include or consist of a mechanical switch, a foil switch, a touch switch, a magnetic switch, and/or a proximity switch. More preferably, the switch is a mechanical switch.

The switch may be adapted to be engaged directly by the user (for example in case of a touch switch) and/or by relative movement between different components of the drug delivery device.

The use signal may comprise and/or result from a change of an electrical resistance, capacity, and/or inductivity of the switch. The use signal may comprise an electrical signal, a change in an electric signal, a digital signal, and/or a change in a digital signal, for example.

According to one aspect, the electronic system is configured to wake up (for example, from a sleep state of the electronic system) upon the use signal. Additionally or alternatively, the electronic system is configured to switch to the measurement state upon the use signal.

In order for dosing operations to be accurately detected, the electronic system must be activated before the specific movement of the mechanism occurs or at least by the occurrence of the specific movement. This can be achieved via the means of the switch. In particular, the sensor arrangement must be operated in the course of the dosing operation. In other words, the switch may trigger wake up (start-up) of the electronic control unit, in more detail wake-up of a main microcontroller. The switch can be monitored by the electronic control unit, in more detail by the main microcontroller, via at least one interrupt. This allows the electronic control unit to detect the occurrence of the specific movement in an easy and energy-efficient manner even when the electronic system is in a sleep state. It is not necessary to permanently operate the sensor arrangement in the sleep state. This saves electrical energy.

Preferably, the sample rate in the measurement state is at least 100 Hz and/or at least the base sample rate. This ensures that the specific movement of the first member relative to the second member is sufficiently measured and described in detail even if the dosing operation is fast.

In one embodiment, the electronic system (preferably specifically the electronic control unit) comprises the main microcontroller and the sensor controller.

According to a further aspect, the electronic system (especially the electronic control unit) may be configured such that the sensor controller operates the sensor arrangement in the measurement state and that the sensor controller is in a power-saving condition else. The power-saving condition may include or consist of a sleep mode of the sensor controller and/or a shut-off state of the sensor controller. In other words, the electronic system may be configured such that the sensor controller is awake (operated or switched on) only in the measurement state.

The main microcontroller may be configured to control the logic flow and the functional behaviour of the electronic system. This may include hardware input and user interface aspects (for example the switch, buttons, and/or LEDs), power management, etc. The sensor controller may be an ultra-low power, low functionality processing core. It may be solely responsible for controlling the sensor arrangement (operating the sensor arrangement) in the measurement state. In addition, the sensor controller may be configured to determine the size of the dose based at least on the sensor data and/or to determine errors occurring in the measurement state. The memory may be accessible for both the main microcontroller and the sensor controller.

The electronic system might be configured such that the main microcontroller starts the sensor controller (preferably only) upon the use signal from the switch. The main microcontroller may be also adapted to configure the sensor controller when the dosing operation starts. The electronic control unit may continue the measurement state until it determines that dosing operation has finished. The sensor controller may finish operation after dosing operation has been completed.

The sensor controller may store the sensor data, measurement results determined on the basis of the sensor data, and/or associated diagnostic information in the memory.

According to a further aspect, the main microcontroller is not used for operating the sensor arrangement in the measurement state.

In one embodiment, the sensor arrangement is operable to provide sensor data indicating transitions between subsequent positions of the first member relative to the second member. The specific movement may cause transitions between the subsequent positions. In particular, the sensor arrangement may be operable to provide sensor data indicating transitions between subsequent positions of the first member relative to the second member caused by the specific movement. Hence, the sensor arrangement can detect and describe the specific movement. A number of consecutive transitions may be indicative of an extent of specific movement. An overall number of consecutive transitions during dosing operation may be indicative of the overall specific movement during the respective dosing operation and/or of size of the dose of the respective dosing operation. A time interval between consecutive transitions may be indicative of the speed of the specific movement. Hence, it may be indicative of the speed of (the at least one part of) the dosing operation. Especially an overall time for a plurality of consecutive transitions during (the at least one part of) one dosing operation and the corresponding overall number of transitions, and/or an average time interval between consecutive transitions of a plurality of consecutive transitions during (the at least one part of) one dosing operation may be indicative of the (average) speed of (the at least one part of) the respective dosing operation.

According to one aspect, the sensor arrangement may be operable to provide sensor data that allows (sensor readings that allow) the electronic system to distinguish between different positions of the first member relative to the second member, for example between at least two successive relative rotational positions (along the specific movement), more preferably between at least four successive relative positions. If a first sensor reading indicates a first relative position and a later second sensor reading indicates a subsequent second relative position, a transition has occurred. Typically, measurement data may comprise a plurality of individual sensor readings, each sensor reading indicating one of the different positions.

In one embodiment, the electronic system (for example the electronic control unit, preferably specifically the sensor controller) is configured to calculate the speed of the specific movement during (the at least one part of) the dosing operation based at least on the sensor data. In other words, the electronic system not only checks whether the speed of the specific movement breaches the first speed threshold (and/or additional speed thresholds) but actually calculates the speed of the specific movement and/or the dosing operation. As there may be an unambiguous relationship between the speed of the specific movement and the speed of the dosing operation, for example a linear relationship, it is not necessary to distinguish between them. The speed may be calculated in terms of transitions per time unit and/or in units of the fluid (e.g. medicament) per time unit. In other words, the electronic system may be configured to calculate the setting speed and/or the dispense speed, for example.

The electronic system (for example the electronic control unit, preferably specifically the sensor controller) can be configured to determine if the speed of the specific movement during (the at least one part of) the dosing operation breaches the first speed threshold (and/or additional speed thresholds) based at least on the calculated speed. If the speed is calculated anyway, it can be used for checking whether the threshold(s) are breached as well.

According to one aspect, the first speed indication can include or consist of the calculated speed. Similarly, the first speed warning can include or consist of making the calculated speed perceptible, for example by displaying it on a display.

In one embodiment, the detection whether the speed of the specific movement breaches the first speed threshold is made only for a part (a portion) of the dosing operation. The same may apply with regard to the at least one further speed threshold explained above. Additionally or alternatively, the electronic system can be configured to calculate the speed of the specific movement only during the part (the portion) the dosing operation.

For example, the detection(s) and/or the speed calculation may be performed only for an initial portion of the dosing operation (for example dose delivery operation), only for an end portion of the dosing operation, and/or only for an intermediate portion between the beginning portion and the end portion. On the one hand, this does not exclude that the electronic system is configured to operate the sensor arrangement during the complete dosing operation to provide sensor data. For example, the electronic system may use the sensor data of the complete dosing operation for determining the size of the dose. On the other hand, it is also possible that the electronic system is configured to operate the sensor arrangement during the dosing operation only during the part of the operation to provide sensor data. Apart from the above, this does not exclude that the electronic system (specifically the main microcontroller) may operate the sensor arrangement for other purposes when not being in the measurement state.

Naturally, the detection(s) and/or the speed calculation may be also performed during the complete dosing operation or during several portions of the dosing operation.

According to one aspect, the electronic system (in particular the electronic control unit, for example the sensor controller) may be configured to calculate the speed by any one of, several of, or all of the following:
Measuring a time to dispense a certain number of units (of the fluid, e.g. the medicament). The certain number of units may be one or multiples thereof.
Measuring a number of units dispensed in a certain time.
Measuring a time to dispense a certain number of units and taking an average over all the measurement periods over a portion of or the complete dispense (i.e. over the at least one part of or complete dose delivery operation). The certain number of units may be one unit or multiples thereof.
Measuring a number of units dispensed in a certain time and taking an average over all the measurement periods over a portion of or the complete dispense.
Measuring a total time and a total number of units dispensed (i.e. during complete dose delivery operation) and calculating the overall average speed.
Measuring a time to dispense a certain number of subsequent units, with a ‘block’ of the certain number of subsequent units constantly moving as a total number of units increases. The certain number of units may be one or multiples thereof.
Measuring a number of units dispensed in a certain time period, with the ‘block’ of the certain time period considered constantly moving as the total number of units increases.

The respective certain values may be pre-defined. They may be pre-programmed and/or stored in the memory.

A decision to provide a corresponding speed warning and/or to include a corresponding speed indication in the data record (the dose record) for the respective threshold (for example to provide the first speed warning and/or to include the first speed indication in the data record (dose record) for the first speed threshold) may be based upon any one or several of the following:
A maximum speed calculated being above or below the respective threshold.
A minimum speed calculated being above or below the respective threshold.
A median speed calculated being above or below the respective threshold.
A certain percentile speed calculated being above or below the respective threshold.
An average speed calculated being above or below the respective threshold.
An alternative formula or algorithm utilising the calculated speed(s) and the respective threshold.

According to a further aspect, the electronic system (in particular the electronic control unit, for example the sensor controller) may be configured to determine that the speed of the specific movement during (the at least one part) of the dosing operation exceeds the first speed threshold if the sensor data indicates that
a time interval between consecutive transitions is below a (pre-defined) single transition time threshold,
a time interval between a certain number of transitions is below a (pre-defined) multiple transitions time threshold, and/or
a number of transitions in a given time interval is greater than a (pre-defined) maximum transition rate.

Similarly, the electronic system (in particular the electronic control unit, for example the sensor controller) may be configured to determine that the speed of the specific movement during (the at least one part) of the dosing operation falls below the first speed threshold if the sensor data indicates that
a time interval between consecutive transitions is above a (pre-defined) single transition time threshold,
a time interval between a certain number of transitions is above a (pre-defined) multiple transitions time threshold, and/or
a number of transitions in a given time interval is less than a (pre-defined) minimum transition rate.

All the above approaches allow easy, reliable, and accurate determination whether the specific movement during (the at least one part of) the dosing operation breaches the first speed threshold. The single transition time threshold, the multiple transitions time threshold, the maximum transition rate, and/or the minimum transition rate may be pre-programmed and/or stored in the memory, for example. Naturally, every one of the approaches can be applied with regard to the at least one further speed threshold(s) accordingly, if any.

In a preferred embodiment, the dosing operation includes or consists of a dose delivery operation for delivering a dose (of the medicament) by the drug delivery device, wherein the size of the dose to be delivered is user-settable or pre-defined. More preferably, the dosing operation is the dose delivery operation. This is particularly advantageous as the risk for excessive speeds is particularly high for dose delivery operations. In addition, high speed during dose delivery operation increases the risk that the injection is painful for the patient.

According to a further aspect, the “dispense speed” during dose delivery operation may correspond, for example (at least substantially) linearly or inversely, to a speed of the specific movement during dose delivery operation. The dispense speed may be the speed with which the dose is actually expelled (for example indicated units of the medicament per time unit), and/or an actual speed of a piston rod during dose delivery operation.

Preferably, the electronic system and/or the second device comprise(s) a conversion function to convert the speed of the specific movement during dose delivery operation to the dispense speed. In embodiments, in which the dispense speed does not correspond linearly to the specific movement during dose delivery operation, said conversion function may be non-linear. The conversion is particularly beneficial if the speed is presented to the user, for example by the electronic system and/or the second device. Presenting the dispense speed might be more intuitive for the user than presenting the speed of the specific movement.

Alternatively or additionally, the dosing operation includes or consists of a dose setting operation for setting the size of the dose to be delivered by the drug delivery device. As the size of the dose delivered by the dose delivery operation is determined by the dose setting operation, the size of the dose can be already measured during dose setting operation as well. Naturally, the limitations of the sensor arrangement, for example maximum time resolution, apply with regard to dose setting operation. Furthermore, particularly slow setting speed indicates an increased risk that the user was doubtful and/or distracted.

A “setting speed” during dose setting operation may correspond, for example (at least substantially) linearly or inversely, to a speed of the specific movement of the first member relative to the second member during dose setting operation. The setting speed may be the speed with which the size of the set dose increases/decreases. The electronic system and/or the second device can comprise a conversion function to convert the speed of the specific movement during dose setting operation to the setting speed. In embodiments, in which the setting speed does not correspond linearly to the specific movement during dose setting operation, said conversion function may be non-linear.

In one embodiment, the overall specific movement of the first member relative to the second member during dosing operation (for example during dose delivery operation) corresponds to the size of the dose, wherein the electronic system (for example, the electronic control unit) is configured to determine the size of the dose based at least on the sensor data obtained by operating the sensor arrangement during the dosing operation. This allows the electronic system to automatically record, provide, store, and/or transmit the size of the dose. This is particularly advantageous for drug delivery devices in which the size of the dose is user-settable and manually set by the user.

According to one aspect, the specific movement includes or consists of a (specific) rotation of the first member relative to the second member during the dosing operation. In other words, the first member rotates relative to the second member during the dosing operation. The (specific) rotation may be only along a specific rotational direction, for example only clockwise or only anti-clockwise. More preferably, the (specific) rotation occurs only during the dosing operation. In a highly preferred embodiment, the dosing operation is dose delivery operation and the (specific) rotation occurs only during dose delivery operation. For example, the first member does not rotate relative to the second member during dose setting operation. Alternatively, the first member may rotate relative to the second member during dose setting operation in a rotational direction opposite to the specific rotational direction. The dose delivery device, in particular the dose setting and drive mechanism thereof, may be adapted in this manner. other words, the first member rotates relative to the second member during the dosing operation.

Preferably, the drug delivery device is configured such that the specific movement occurs only in one direction in usual operation, for example only along the specific rotational direction. Usual operation may include at least dose setting operation and dose delivery operation. In case the drug delivery device is a reusable device, resetting the drug delivery device in conjunction with replacing an empty cartridge with a new, full cartridge may be not part of usual operation in this regard.

Any “backward” transitions (transitions in a direction contrary to the one direction) indicate a decrease in the size of the dose, especially after dose delivery operation has begun. Hence, backward transitions suggest a mechanical and/or electrical failure, or momentary oscillations in a reverse direction, so-called ‘jitter’.

In one embodiment, the electronic system, for example the electronic control unit, only considers “forward” transitions (related to increase of the size of the dose) for detecting whether the speed of the specific movement during (the at least one part of) the dosing operation
breaches the first speed threshold and/or
breaches the at least one further speed threshold.

Hence, jitter and/or backward transitions cannot incorrectly affect the detection whether the respective speed threshold is breached.

In one embodiment, the electronic system considers only forward transitions within (the at least on part of the) the respective dose operation for the speed calculation. Hence, jitter and/or backward transitions cannot incorrectly affect the speed calculation.

Accordingly, the electronic control system, for example the electronic control unit, may be adapted to determine an error if the sensor data for the dosing operation (particularly for the dose delivery operation) includes
any backward transition and/or
at least a certain number of backward transitions.

It may be further adapted to include a corresponding error indication in the data record for the respective dosing operation, for example by raising a corresponding flag or by setting a corresponding specific purpose value.

In one embodiment, the electronic system (preferably the electronic control unit) is configured to generate the dose record for the respective dosing operation. The dose record may include the size of the dose and a time stamp of the dose. Additionally or alternatively, the dose record may include the diagnostic information. In other words, the dose record may include other and/or additional data depending on pre-defined circumstances. The electronic system may be configured to store the dose record in the memory, and/or for transmission of the dose record via the communication unit, e.g. to the second device. The dose record may be generated automatically at the end of the measurement state. A dose record pattern for the dose record may comprise or consist of at least a time stamp field, which is at least suitable for storing the time stamp of the dose, and/or a dose size field, which is at least suitable for storing the size of the dose.

In one embodiment, the dose record constitutes or includes the data record. In other words, the electronic system (preferably the electronic control unit) includes all data explained with regard to the data record in the dose record. In another embodiment, the data record is provided as a separate record for the respective dosing operation.

The first speed indication may be set in as a corresponding specific purpose value in the dose record for the respective dosing operation
in the time stamp field instead of the time stamp and/or
in the dose size field instead of the size of the dose
   depending on whether the specific movement during (the at least one part of) the dosing operation breaches the first speed threshold.

For example, the corresponding specific purpose value may be set if the specific movement during (the at least one part of) the dosing operation
exceeds the first speed threshold,
(in another embodiment) does not exceed the first speed threshold,
(in still another embodiment) falls below the first speed threshold, or
(in still another embodiment) does not fall below the first speed threshold.

By storing and/or transmitting the dose record, the dose record is made available for later evaluation, for example by the electronic system itself, by the user, the HCP, the manufacturer, and/or the second device. Storing the dose record allows the electronic system to transmit the dose record later if the second device is not paired and/or connected to the electronic system at the moment. In particular, the electronic system may be configured to store a plurality of dose records in the memory, for example at least the ten latest data records, more preferably at least the latest fifty data records.

The synchronization state mentioned above may be a state for transferring data from the electronic system to the second device and/or for transferring data from the second device to the electronic system. In particular, the synchronization state may be adapted for transmission of data records and/or dose records to the second device. In more detail, the synchronization state may be adapted for completing, updating and/or synchronizing data records and/or dose records stored in a memory of the second device with at least one dose record provided by the electronic system. The electronic system and/or the second device may be configured accordingly. The at least one dose record provided by the electronic system may include the dose record provided as a result of a presently finished dose delivery operation and/or at least one dose record stored in the memory. Preferably, the electronic system automatically switches to the synchronization state after dose delivery operation has finished. For example, the electronic system may be configured to switch to the synchronization state when it switches out of the measurement state. Preferably, the electronic switches to the synchronization state automatically only if a new data record and/or dose record is provided.

The electronic system, especially the sensor arrangement, may include at least one sensor. The sensor(s) may be one or more of any one of, several of, or all of the following sensor means: accelerometers, light sensors, sound sensors, pressure sensors, temperature sensors, proximity sensors, infrared sensors, ultrasonic sensors, colour sensors, humidity sensors, tilt sensors, flow sensors, magnetic/Hall effect sensors, radiation sensor, lidar, electrical current sensors, optical sensors, force/torque sensors, strain gauges, and/or mechanical switches. Any one of the sensors may be digital and/or analogue, may include digital to analogue conversion and/or vice versa. Each sensor may be operatively connected to the electronic control unit. The electronic control unit may be configured to operate the sensor(s), for example at least during (the at least one part of) the dosing operation.

Each sensor may be configured to distinguish between two states, wherein the two states may depend on the position of the first member relative to the second member.

In one embodiment, the sensor arrangement comprises at least one of a light source with a corresponding optical sensor, an electrical sliding contact sensor, a mechanical switching arrangement, an inductive sensor, and a magnetic sensor.

More preferably, the sensor arrangement may include at least two sensors. Especially, the sensor arrangement may comprise at least one light source and at least two optical sensors, wherein the sensor arrangement is configured to provide sensor data allowing to distinguish at least four different (successive) positions of the first member relative to the second member.

The sensor arrangement may be configured to form, together with an encoder component, a motion sensor system (at least in a final configuration). In other words, the sensor arrangement is a sensor arrangement for the motion sensor system. The motion sensor system comprises (or consists of) the sensor arrangement and the encoder component. The electronic system comprises at least the sensor arrangement of the motion sensor system. The sensor arrangement may be adapted to use the encoder component for generating the sensor data.

In one embodiment, the specific movement includes or consists of an axial movement and the motion sensor system is a linear motion sensor system. The first member may be a piston rod or a drive sleeve for driving the piston rod that axially moves during dose delivery operation, for example.

In the case that the specific movement includes or consists of the (specific) rotation, the sensor arrangement may be configured to distinguish different (successive) rotational positions of the first member relative to the second member. The motion sensor system may be a rotary sensor system. The sensor arrangement may be at least rotationally fixed with regard to the second member. Especially, the sensor arrangement may be at least rotationally fixed (directly) to the second member. The encoder component may be at least rotationally fixed with regard to the first member. Especially, the encoder component may be rotationally fixed to the first member.

The sensor arrangement may be axially fixed with regard to the second member, preferably (directly) to the second member. The sensor arrangement may be formed integrally with the second component. The encoder component may be axially fixed with regard to or (directly) to the first member. The encoder component may be formed integrally with the first component.

The encoder component may comprise a plurality of spatially separated detection regions. In case of a rotary sensor system, the encoder component may comprise a plurality of angularly separated detection regions. Regions between two adjacent detection regions may be referred to as non-detection regions. In one embodiment, the encoder ring only comprises the detection regions and the non-detection regions are formed by the dial second member, for example the dial sleeve. The encoder ring may be clipped to the dial sleeve.

The detection regions may differ from the non-detection regions in at least one property that can be detected by the at least one sensor, for example in an optical property (such as a reflectivity and/or colour), in a magnetic property, an electrical property (such as conductivity), and/or any other detectable property. In one embodiment, the detection regions exhibit a higher reflectance for the radiation emitted from the light source(s) than the non-detection regions. The detection regions may exhibit a high reflection for the radiation and the non-detection regions may exhibit a lower or no reflection for the radiation. In one exemplary embodiment, the high reflection regions are white and the non-detection regions are black. The encoder ring and especially the detection regions may be moulded with an IR reflective additive.

Each sensor may be configured that at least two states can be distinguished, for example a first state (binary 0) and a second state (binary 1), white and black, and/or another combination of two different states. In one embodiment, the respective individual sensor indicates the first state when it faces one of the non-detection regions and the second state when it faces one of the detection regions of the encoder component.

It may depend on the position of the first member relative to the second member whether the individual sensor faces one of the detection regions or one of the non-detection regions. Accordingly, it may depend on the relative position of the first member relative the second member whether the individual sensor indicates the first state or the second state. When the position changes due to the specific movement, the individual sensor indicates a change (also referred to as a transition), for example from the first state (such as binary 0) to the second state (such as binary 1) or vice versa. For example, a transition occurs when an edge between one of the detection regions and an adjacent non-detection region passes the respective sensor. In one embodiment, it is interpreted that each transition corresponds to an increase or decrease of the size of the dose by a single unit (of the fluid, e.g. the medicament). In more general, each transition may be interpreted as a multiple of a single unit, or a fraction of a single unit, depending on the configuration of the system.

If there are (at least) two sensors, a detection pattern (including the states of all of the sensors) of the sensors depends on and changes with the position of the first member relative to the second member. Preferably, the sensors and the detection regions are arranged such that identical transitions of the individual sensors resulting from the specific movement are offset to each other. In other words, identical transitions (for example from the first state to the second state) of two different sensors do not occur at the same time and/or at the same position of the first member relative to the second member. This allows the electronic system to detect additional information, such as distinguishing between a “forward” and a “backward” movement of the first member relative to the second member. Accordingly, it is also possible to determine (for example by the electronic control unit) whether transitions indicate an increase or decrease of the size of the dose. In one embodiment, the electronic system considers only increasing edge-to-edge transitions (“forward” transitions) corresponding to “forward” specific movement increasing the size of the dose for the detection whether speed threshold(s) are breached and/or for determining the size of the dose.

Configurations, whereby the sensors indicate one of two states and transitions occur between these states are applicable to any of the sensor technologies indicated above.

In one embodiment, the sensor arrangement is configured to provide the sensor data corresponding to a Gray code. In more detail, the individual sensor readings may be provided corresponding to a Gray code.

Preferably, the sensor arrangement (and optionally the whole motion sensor system) may be implemented according to any of the embodiments described in WO 2019/101962 A1, unpublished EP 20315357.2, EP 20315066.9, EP 20315451.3, and EP 21315002.2. More preferably, the sensor arrangement (and optionally the whole motion sensor system) is implemented according to any of the embodiments described in unpublished EP 20315305.1.

The encoder component may be a part of the second member, fixed to, or fixed relative to the second member.

It may be considered that the encoder component is not part of the electronic system as such. In one embodiment, the encoder component (as such) does not include electrical parts (including electronic parts) of the electronic system. In particular, the sensor arrangement may comprise all active parts for the motion sensor system, for example all electric and/or electronic parts. The encoder component may be a purely passive part. According to another aspect, the encoder component may be regarded as part of the electronic system. In other words, the electronic system comprises the whole motion sensor system including the sensor arrangement and the encoder component.

The electronic system may comprise a display. For example, the display may be adapted to display any one of, several of, and/or all of the following:
The first speed warning,
the (calculated) speed,
one of, several of, and/or all of the at least one additional speed warning(s),
the size of the dose delivered by the last dose delivery operation,
date and/or time information regarding the last dose delivery operation,
dose records,
a present time,
a status information regarding the status of the electrical power supply, for example a battery status such as an indication if the electrical power supply is low,
an indication that the electronic system is in the measurement state,
an indication that the electronic system is in the synchronization state, and
an indication that the electronic system is in the pairing state.

The electronic system may comprise an LED indicator connected to the electronic control unit. The LED indicator may include at least one indicator LED. Different indicator LEDs may emit different colours of light.

The electronic system may be configured to show warnings and/or alerts using the LED indicator, for example the first speed warning. Alternatively or additionally, the electronic system may be adapted to indicate when the electronic system is in at least one certain state via the LED indicator. For example, the LED indicator may unambiguously indicate when the electronic system is in the measurement state and/or when the electronic system is in the pairing state. Different indications can differ from each other by use of different colours of light, by different spatial illumination patterns and/or by different illumination pattern sequences in time.

In one embodiment, the electronic system comprises a sound generator. The sound generator may be part of or operatively connected to the electronic control unit. The electronic system may be adapted to audibly provide the first speed warning and/or at least one further speed warning (e.g. the second speed warning). Audibly providing any one of the speed warnings may include a corresponding warning sound, a corresponding speech warning, audibly indicating the calculated speed (for example by reading aloud the calculated speed), audibly indicating information regarding potential causes for the breach, and/or audibly indicating instructions and/or suggestions for the user, for example in order to change their dosing operation behaviour. The warning sounds and the corresponding speech warning may be different for (at least some of) the different speed thresholds.

In one embodiment, the electronic system comprises a clock generator for providing date and time information. The clock generator may comprise or consist of a real time clock. This ensures accurate, consistent, and reliable date and time information. The clock generator may be part of the electronic control unit or operatively connected to the electronic control unit. The electronic control unit may be configured to operate the clock generator for determining a time stamp of the dose. The clock generator may also be used for determining the sample rate and/or time intervals.

According to one aspect, the data record and/or the dose record may include at least one flag field for storing at least one corresponding flag, for example the first speed flag field. Each flag field consists of at least one bit. Any one of the at least one flag field may consist of several bits, for example of two bits, three bits, or four bits. Preferably, each flag field consists of one bit if nothing else is stated. Flag fields may be used to indicate certain information regarding the dosing operation, the dose delivered, and/or the status of the device.

In one embodiment, the electronic system comprises at least one further speed threshold (i.e. in addition to the first speed threshold), for example at least a second speed threshold.

Preferably, the electronic system is configured to determine, based on the sensor data, if a speed of the specific movement during (the at least one part of) the dosing operation breaches the at least one further speed threshold.

The embodiments, modifications, and advantages described with regard to the first speed threshold, the first speed indication, and the dosing operation may apply accordingly with regard to the at least one further speed threshold.

In one embodiment, the at least one further speed threshold is less than the first speed threshold.

Especially, in one embodiment, the problem indicated above is solved by an electronic system for a drug delivery device, in which a first member performs a specific movement relative to a second member during a dosing operation,
wherein the electronic system comprises a sensor arrangement, which is operable to provide sensor data describing the specific movement, and wherein the electronic system is configured to operate the sensor arrangement during at least one part of the dosing operation to provide sensor data,
wherein the electronic system is (at least) configured to determine, based on the sensor data, if a speed of the specific movement during the at least one part of the dosing operation breaches a first speed threshold,
wherein the electronic system comprises at least one further speed threshold and is configured to determine, based on the sensor data, if the speed of the specific movement during the at least one part of the dosing operation breaches the at least one further speed threshold, wherein the at least one further speed threshold is less than the first speed threshold.

The modifications and advantages described with regard to other embodiments apply, accordingly, for this embodiment and vice versa.

The at least one further speed threshold old may be pre-programmed and/or stored in the memory.

According to a further aspect, the electronic system may be configured to provide the at least one further speed warning (e.g. at least a second speed warning) depending on whether the specific movement during the at least one part of the dosing operation breaches the at least one further speed threshold. Providing the at least one further speed warning may include
generating a visual, audible, and/or tangible alert (for a user), and/or
transmitting an at least one further speed warning signal by means of the communication unit, for example to the second device.

Additionally or alternatively, the electronic system (especially the electronic control unit) may be configured to generate the data record (which may be the dose record, part of the dose record or provided in addition to the dose record) including an at least one further speed indication (e.g. a second speed indication) for the dosing operation depending on whether the specific movement during (the at least one part of) the respective dosing operation breaches the at least one further speed threshold. By this, the at least one further speed indication for the respective dosing operation can be provided and/or stored. For example, the at least one further speed indication may include or consist of a corresponding flag and/or a corresponding specific purpose value. The at least one further speed indication may be set in as a corresponding specific purpose value in the dose record for the respective dosing operation
in the time stamp field instead of the time stamp and/or
in the dose size field instead of the size of the dose
   depending on whether the specific movement during (the at least one part of) the dosing operation breaches the at least one further speed threshold.

According to another aspect, a corresponding flag may be raised in the data record (the dose record) in case the specific movement during (the at least one part of) the respective dosing operation exceeds the at least one further speed threshold. In more detail, the data record (the dose record) may include a corresponding flag field, wherein said flag field is set to a (first) value corresponding to ‘true’ in this case but set to a (second) value corresponding to ‘false’ else. In another embodiment, the at least one further speed flag is raised in case the specific movement during (the at least one part of) the respective dosing operation falls below the second speed threshold.

In one embodiment, the first speed threshold or (one of) the at least one further speed threshold(s) is a very fast speed threshold. Preferably, the very fast speed threshold is the first speed threshold. The very fast speed threshold may be an upper threshold.

The very fast speed threshold may correspond to
the maximum time resolution and/or the first sample rate or
to a certain proportion to the maximum time resolution and/or the first sample rate.

Especially, in one embodiment, the problem indicated above is solved by an electronic system for a drug delivery device, in which a first member performs a specific movement relative to a second member during a dosing operation,
wherein the electronic system comprises a sensor arrangement, which is operable to provide sensor data describing the specific movement, and wherein the electronic system is configured to operate the sensor arrangement during at least one part of the dosing operation to provide sensor data,
wherein the electronic system is (at least) configured to determine, based on the sensor data, if a speed of the specific movement during the at least one part of the dosing operation breaches a first speed threshold,
wherein the first speed threshold is a very fast speed threshold, which corresponds to
   a maximum time resolution of the sensor arrangement and/or the first sample rate or
   to a certain proportion to the maximum time resolution and/or the first sample rate.

The modifications and advantages described with regard to other embodiments apply, accordingly, for this embodiment and vice versa.

The term “maximum time resolution and/or the first sample rate” may be referred to as “applicable measurement capability”. It is explained elsewhere that speed thresholds can be defined in different terms (e.g. as single transition time threshold, multiple transition time threshold, average transition time threshold, maximum transition rate, dispense speed value, etc.). The proportion may be less than 100%, preferably less than 50%, most preferably less than 35%. As it may be impossible to accurately detect speeds beyond the applicable measurement capability, the very fast speed warning and/or indication preferably should be provided well below the applicable measurement capability is actually reached. This ensures that exceeding the very fast speed threshold is detected well before the applicable measurement capability is actually reached. Consequently, the corresponding speed warning (a very fast speed warning) and/or the corresponding speed indication (a very fast speed indication) is/are provided before the applicable measurement capability is actually reached or exceeded. The patient, the user, the HCP, the manufacturer, and/or the second device will be made aware of the problem and can change their behaviour or alert to change the behaviour before the speed of the dosing operation actually exceeds the applicable measurement capability.

Additionally or alternatively, the proportion may be at least 10% of the speed corresponding to the maximum time resolution, preferably at least 15%, more preferably at least 20%. This prevents that the patient, the user, the HCP, the manufacturer, and/or the second device are unnecessarily warned, unsettled, confused, and/or bothered.

According to another aspect, the very fast speed threshold is set to a value which is sufficiently high that the user will not exceed the corresponding speed in normal dosing operation. For example, the very fast speed threshold may correspond to a speed above a maximum speed that is likely achievable by the user. For example, the very fast speed threshold corresponds to a speed higher than the expected maximum speed explained above. In one embodiment, the very fast speed threshold corresponds to at least 1.1 times the expected maximum speed for normal operation.

Especially, in one embodiment, the problem indicated above is solved by an electronic system for a drug delivery device, in which a first member performs a specific movement relative to a second member during a dosing operation,
wherein the electronic system comprises a sensor arrangement, which is operable to provide sensor data describing the specific movement, and wherein the electronic system is configured to operate the sensor arrangement during at least one part of the dosing operation to provide sensor data,
wherein the electronic system is (at least) configured to determine, based on the sensor data, if a speed of the specific movement during the at least one part of the dosing operation breaches a first speed threshold,
wherein the first speed threshold is a very fast speed threshold, which is set to a value which is sufficiently high that a user will not exceed the corresponding speed in normal dosing operation.

The modifications and advantages described with regard to other embodiments apply, accordingly, for this embodiment and vice versa.

For example, the very fast speed threshold may be a single transition time threshold. In other words, the very fast speed threshold defines a minimum acceptable time interval between two subsequent transitions during the dosing operation. The very fast speed threshold may be in a range from 0.05 ms to 70 ms, preferably from 0.1 ms to 50 ms, more preferably between 0.3 ms and 5 ms. For example, the very fast speed threshold may be defined as single transition time threshold of 1.0 ms±0.5 ms. If the time interval between two subsequent transitions during (the at least one part of) dosing operation is shorter than the single transition time threshold, the speed of the specific movement during (the at least one part of) the dosing operation exceeds the very fast speed threshold. The very fast speed threshold may apply for dose delivery operation (very fast dispense threshold) and/or for dose setting operation (very fast setting threshold).

Additionally or alternatively, the very fast speed threshold may correspond to a certain dispense speed and/or setting speed. Preferably, the very fast speed threshold corresponds to a dispense speed value and/or setting speed value in the range from 200 units (of medicament, for example insulin) per second to 3000 units/s, more preferably from 500 units/s to 2000 units/s, most preferably from 800 units/s to 1200 units/s.

The very fast speed threshold is set to a value which is sufficiently high that the user will not exceed the corresponding speed in normal dosing operation.

Preferably, the electronic system (maybe specifically the electronic control unit) is configured to raise a “very fast speed flag” in the data record (the dose record) if the speed during (the at least one part of the) the respective dosing operation has exceeded the very fast speed threshold, for example by setting a value in a “very fast speed flag field” in the data record (the dose record) to a value corresponding to ‘true’ in this case and to a value corresponding to ‘false’ else (i.e. the very fast speed flag is not raised else). Additionally or alternatively, the electronic system (maybe specifically the electronic control unit) may be configured to set the time stamp field and/or the dose size field (preferably only the dose size field) to a specific purpose value indicating that the speed during (the at least one part of) the respective dosing operation has exceeded the fast speed threshold.

A dose record including the very fast speed indication may be excluded from further evaluation and/or consideration, for example in a titration process.

In one embodiment, electronic system comprises a fast speed threshold. In other words, the first speed threshold or (one of) the at least one further speed threshold(s) is the fast speed threshold. Preferably, the fast speed threshold is one of the at least one further speed threshold(s), for example the second speed threshold. The fast speed threshold may be an upper threshold.

The fast speed threshold may apply for dose delivery operation (i.e. a fast dispense speed threshold). There is an increased risk that the injection is painful for the patient if the dispense speed is high, even if the dispense speed is considerably lower than the very fast dispense speed threshold. Providing the corresponding warning (a fast speed warning) and/or providing the corresponding indication (a fast speed indication, for example a raised fast speed flag) in the data record (the dose record) helps to support the training of the injection behaviours of patients, health care professionals (HCP), and other users in order to limit pain due to unfavourable dispense speed.

Fast dispense speed also indicates a risk that there may have been a “priming gap” between the dose setting and drive mechanism (for example of a piston rod thereof, may be a bearing or foot at a distal end of the piston rod) and the bung of the container. The dispense speed is likely to be higher than “normal” in case of the priming gap, particularly at the beginning of dose delivery operation. The priming gap reduces the actual size of the dose delivered below an intended size of the dose. The intended size of the dose may have been set by dose setting operation or may be pre-defined. In addition, it might be favourable to keep the dispense speed for a certain medicament below the fast speed threshold for medical reasons.

Further, fast dispense speed can indicate that gas is being dispensed, for example if gas (e.g. air) is within the cartridge and at least partly dispense instead of the fluid (e.g. the medicament). A flow resistance through a needle may be less for air than for the fluid. In addition, fast dispense speed can indicate that the cartridge is not mounted. As a consequence, the resistance for advancement of the piston rod is lower and hence higher dispense speed than expected in normal operation. Apart from this, fast dispense speed can indicate that the needle does not penetrate the user's skin. Dispense into air might exhibit less resistance than dispense into the user's body such that higher dispense speed than expected in normal operation may also occur in this case.

For example, the fast speed threshold may be a multiple transitions time threshold. In other words, the fast speed threshold defines a minimum acceptable time for a pre-defined number of transitions. The pre-defined number may be at least three. Preferably, the pre-defined number is in the range from 3 to 15, more preferably in the range from 6 to 10, for example 8. Said time interval (e.g. for 8 transitions) may be in a range from 70 ms to 300 ms, preferably from 90 ms to 200 ms, more preferably between 120 ms and 160 ms. For example, the fast speed threshold may be defined as (minimum acceptable) time interval of 140 ms+4 ms for 8 transitions. If the time interval for 8 subsequent transitions during (the at least one part of) dosing operation is shorter, the speed of the specific movement during (the at least one part of) the dosing operation exceeds the fast speed threshold.

Additionally or alternatively, the fast speed threshold may correspond to a certain dispense speed. Preferably, said certain dispense speed is in the range from 20 units/s to 150 units/s, for example from 30 units/s to 100 units/s, for example 50 units/s.

Preferably, the electronic system (maybe specifically the electronic control unit) is configured to raise a “fast speed flag” in the data record (the dose record) if the speed during (the at least one part of the) the respective dosing operation, which is preferably dose delivery operation, has exceeded the fast speed threshold, for example by setting a value in a “fast speed flag field” in the data record (the dose record) to a value corresponding to ‘true’ in this case and to a value corresponding to ‘false’ else (i.e. the “fast speed flag” is not raised else). Additionally or alternatively, the electronic system (maybe specifically the electronic control unit) may be configured to set the time stamp field and/or the dose size field (preferably only the dose size field) to a specific purpose value indicating that the speed during (the at least one part of) the respective dosing operation has exceeded the fast speed threshold.

According to a further aspect, the electronic system may be configured to set a value of the calculated dispense speed in a dispense speed field in the dose record, at least if the dispense speed during (the at least one part of) the respective dose delivery operation has been equal to or higher than the fast speed threshold.

The fast (dispense) speed threshold may be adjustably by the HCP and/or the user, preferably within a pre-defined range. Hence, the fast speed threshold can be adapted to the individual pain perception of the patient for fast dispense speeds.

The fast speed warning of the electronic system and/or a fast speed warning provided by the second device based on the fast speed indication includes any one of, several of, or all of the following information:
The user is dispensing faster than recommended for the particular medicament.
The user is dispensing too quickly and may be increasing the risk of a painful injection.
The dispense speed is higher than expected for the delivery of the fluid indicating that no cartridge is fitted, air is dispensed, and/or the needle is not penetrating the patient's skin.

In one embodiment, the electronic system comprises a low speed threshold. In other words, the first speed threshold or (one of) the at least one further speed threshold(s) is the low speed threshold. Preferably, the low speed threshold is one of the at least one further speed threshold(s), for example a third speed threshold. The low speed threshold may be a lower threshold or a higher threshold. In the first case, the underlying idea is that it is unfavourable that the speed falls below the low speed threshold during (the at least one part of) the dosing operation. In the second case, the underlying idea is that it is favourable that the speed exceeds the low speed threshold during (the at least one part of) the dosing operation.

The low speed threshold may apply for dose delivery operation (i.e. a low dispense speed threshold). There is an increased risk that the injection is painful for the patient if the dispense speed is low because the dose delivery operation takes too long. Providing the corresponding warning (a low speed warning) and/or providing the corresponding indication (a low speed indication, for example a raised low speed flag) in the data record (the dose record) helps to support the training of the injection behaviours of patients, health care professionals (HCP), and other users in order to limit pain due to unfavourable dispense speeds.

As explained above, if the dispense speed is slow (particularly slower than expected for the normal (proper) dose delivery operation), this indicates a partially blocked needle, and/or that a viscosity of a dispensed fluid (for example a medicament, e.g. insulin) is higher than expected.

According to an aspect, the low speed threshold may correspond to a speed of the dosing operation in the range from 0.01 units/s to 0.5 unit/s.

A low speed warning of the electronic system and/or a low speed warning provided by the second device based on the low speed indication may include any one of, several of, or all of the following information:
The user is dispensing slower than recommended for the particular medicament.
The user is dispensing too slowly and may be increasing the risk of a painful injection.
The dispense speed is slower than expected for normal operation.
The slow dispense speed indicates a partially blocked needle.
The slow dispense speed indicates that a viscosity of the fluid is higher than expected (this may be an indication for use of a wrong fluid).

Additionally or alternatively, the electronic system may comprise a low setting speed threshold. Slow setting speed and/or dispense speed can indicate that the user is doubtful and/or distracted.

The above-mentioned problem is further solved by a drug delivery device comprising the electronic system according to any one of the embodiments described.

The explanations of embodiments, modifications, and advantages regarding the electronic system apply accordingly concerning the drug delivery device as well, and vice versa.

The drug delivery device may comprise a dose setting and drive mechanism, which is configured to perform (at least in the final configuration) a dose setting operation for setting a dose to be delivered by the drug delivery device and a dose delivery operation for delivering the set dose. The dose setting and drive mechanism comprises the first member and the second member. The size of the dose may correspond to overall specific movement of the first member relative to the second member during the respective dose delivery operation and/or the dose setting operation (preceding the respecting dose delivery operation). For example, the dose delivered by dose delivery operation may linearly depend from said overall specific movement.

The drug delivery device may comprise a housing. In more detail, the dose setting and drive mechanism may include the housing. The housing retains and protects (the further components of) the dose setting and drive mechanism, for example from mechanical damage and dirt.

According to a further aspect, a transition from dose setting operation to dose delivery operation may include that the first member and the second member become rotationally de-coupled with regard to each other.

Preferably, the drug delivery device is a pen-type injector. Additionally or alternatively, the drug delivery device is manually driven device.

The drug delivery device may comprise a container receptacle which is adapted to receive a container containing a medicament. The container receptacle may be permanently or releasably fixed to the dose setting and drive mechanism.

In one embodiment, the dose setting and drive mechanism and/or the second member comprise at least one clutch. The second member and (the other parts of) the dose setting and drive mechanism can form the at least one clutch together. The at least one clutch may be configured such that the second member is rotationally coupled with regard to the first member during dose setting operation and/or that the second member is rotationally de-coupled with regard to the first member during dose delivery operation. The at least one clutch may be configured such that the transition from dose setting operation to dose delivery operation includes that the at least one clutch rotationally de-couples the second member and the first member.

According to another aspect of the present disclosure, the dose setting and drive mechanism comprises a dial sleeve assembly. The dial sleeve assembly may rotate relative to the second member and/or the housing at least during dose delivery operation. The first member may be at least rotationally coupled to the dial sleeve assembly. In particular, the first member may be the dial sleeve assembly or a part of the dial sleeve assembly. In an embodiment, the dial sleeve assembly may not rotate relative to the second member during dose setting operation. In one embodiment, the dial sleeve assembly is configured to rotate relative to the housing during dose setting operation and dose delivery operation. For example, the dial sleeve assembly may move on a helical path with regard to the housing during dose setting operation and dose delivery operation.

The dial sleeve assembly may include or consist of a number sleeve and/or a dial sleeve. The number sleeve and/or the dial sleeve may have a threaded engagement with regard to the housing. For example, the number sleeve may have a threaded engagement with the housing directly or to an insert that is axially and rotationally fixed to the housing. The number sleeve and/or the dial sleeve may comprise an inner thread or an outer thread for threaded engagement with the housing (insert).

The encoder component may comprise or consist of an encoder ring attached to the dial sleeve assembly. In more detail, the encoder ring may be attached to the dial sleeve.

The above-mentioned problem is also solved by a button module for a drug delivery device, wherein the button module is permanently mounted, permanently mountable, or releasably mountable on a main part of to the dose setting and drive mechanism, wherein the button module comprises the electrical system according to any one of the embodiments described and is adapted such that the button module or a part thereof constitutes the second member when mounted on the main part.

The explanations of embodiments, modifications, and advantages regarding the electronic system and the drug delivery device apply accordingly concerning the button module as well, and vice versa.

The main part of the dose setting and drive mechanism may include the first member.

According to one aspect, the button module may be provided separately from the main part of the dose setting and drive mechanism, for example as an individual component.

The drug delivery device, in particular the dose setting and drive mechanism, and/or the electronic system, in particular the motion sensor system, may be only fully functional when the button module is mounted on the main part of the dose setting and drive mechanism (i.e. in a “final configuration”). All described functionalities may at least apply in the final configuration.

Preferably, the button module is the second member.

The button module and/or the electronic control unit may have a distal surface facing towards the main part of the dose setting and drive mechanism, for example for providing an interface for mechanical interaction and/or electrical connection with further component parts. As an example, the distal surface may comprise at least two, e.g. four, contact pads of the electronic control unit which may be selectively connected and disconnected with electronic components, such as switching components.

The button module may be permanently or detachably attached to a trigger, a button, or a dial grip, e.g. at or near the proximal end of the drug delivery device. In the final configuration, the button module may be located at a proximal end of the dose setting and drive mechanism. Especially, the button module may constitute a proximal end of the drug delivery device along the axial direction (at least in the final configuration).

For example, in the device disclosed in EP 2 890 435, the button module may constitute the second member. During dose setting operation, the dial sleeve assembly (e.g. including the number sleeve and the encoder component) and the button module extend (translate) helically from the housing of the device. There is no relative rotation between the button module and the dial sleeve assembly during dose setting operation.

In said embodiment, the button module and the (at least one) clutch are translated distally relative to the housing for transition from the dose setting operation to dose delivery operation. After the clutch has translated a pre-defined distance, e.g. less than 2.0 mm, for example nominally 1.20 mm, the clutch disengages from the dial sleeve and the drug delivery device (in particular the dose setting and drive mechanism) enters a dispensing mode for dose delivery operation. During dose delivery operation, the dial sleeve assembly retracts back along the helical path into the device, whereas the button module does not rotate and only retracts with axial motion, until a zero units stop is engaged and the dose delivery operation is complete. Relative rotation of the button module with respect to the dial sleeve assembly occurs during dose delivery operation. In an exemplary embodiment, the mechanical switch is mounted on an underside (i.e. on a distal side) of the button module and utilises a relative movement between the button module and dial sleeve assembly to trigger, for example the specific movement or a relative movement that occurs during the transition from dose setting operation to dose delivery operation.

Furthermore, the present disclosure relates to a medical system including the electronic system according to any one of the embodiments described, the drug delivery device comprising the electronic system and/or the button module comprising the electronic system.

The second device is configured to receive the dose records from the electronic system. It may be further adapted to store the received dose records in memory of the second device.

The second device may be configured to use the dose records for a dose log and/or a dose helper functionality. For example, the second device may be configured to provide therapy recommendations based on the dose records.

The dose helper functionality may be configured to recommend doses to be set based on the dose records for the patient and measurement values for at least one body property of the same patient, for example blood glucose measurement values. In one embodiment, the dose helper functionality of the second device comprises at least one titration function for stepwise adapting doses of insulin to be set.

The above-mentioned problem is further solved by a method for operating an electronic system for a drug delivery device, preferably the electronic system according to any one of the embodiments described in this disclosed and/or the electronic system of the drug delivery device as described in this disclosure,
wherein a first member of the drug delivery device performs a specific movement relative to a second member of the drug delivery device during a dosing operation,
wherein the electronic system comprises a sensor arrangement, which is operable to provide sensor data describing the specific movement, wherein the electronic system operates the sensor arrangement during (at least one part of) the dosing operation to provide sensor data; wherein the electronic system determines, based on the sensor data,
   if a speed of the specific movement during (the at least one part of) the dosing operation breaches a first speed threshold, and/or
   the speed of the specific movement and/or the dosing operation during (the at least one part of) the dosing operation.

The explanations of embodiments, modifications, and advantages regarding the electronic system, the button module, the dose setting and drive mechanism, the drug delivery device, and the medical system apply accordingly with regard to the method as well, and vice versa. For example, the method may comprise any corresponding steps.

The present invention is particularly applicable for drug delivery devices which are manually driven, e.g. by a user applying a force to the button module, for devices which are driven by a spring or the like, and for devices which combine these two concepts, i.e. spring assisted devices which still require a user to exert an injection force. The spring-type devices involve springs which are preloaded and springs which are loaded by the user during dose selecting. Some stored-energy devices use a combination of spring preload and additional energy provided by the user, for example during dose setting.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e . . . , 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F (ab) and F (ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F (ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The terms “axial”, “radial”, or “circumferential” as used herein may be used with respect to a main longitudinal axis of the device, the cartridge, the housing or the cartridge holder, e.g. the axis which extends through the proximal and distal ends of the cartridge, the cartridge holder or the drug delivery device.

Non-limiting, exemplary embodiments of the invention will now be described with reference to the accompanying drawings, in which:

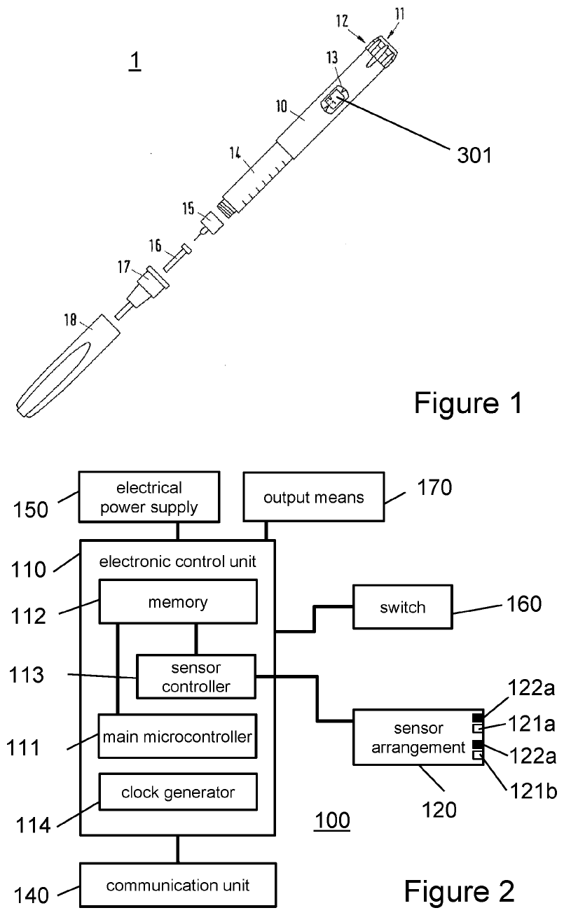

FIG. 1 shows an embodiment of a drug delivery device;

FIG. 2 schematically shows an embodiment of an electronic system according to the present disclosure;

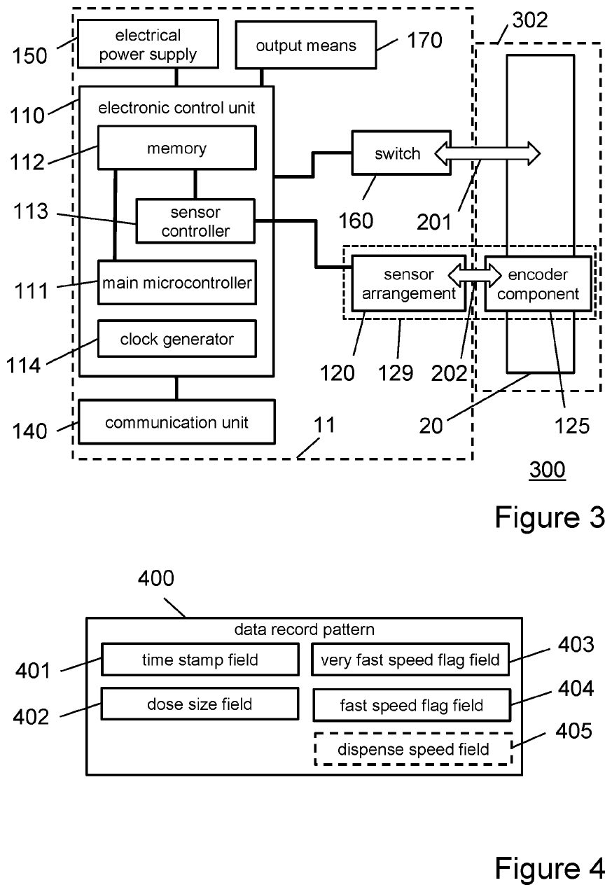

FIG. 3 schematically shows the electronic system of FIG. 2 in a dose setting and drive mechanism of a drug delivery device;

FIG. 4 shows an example of a pattern for dose records provided by the electronic system of FIG. 2;

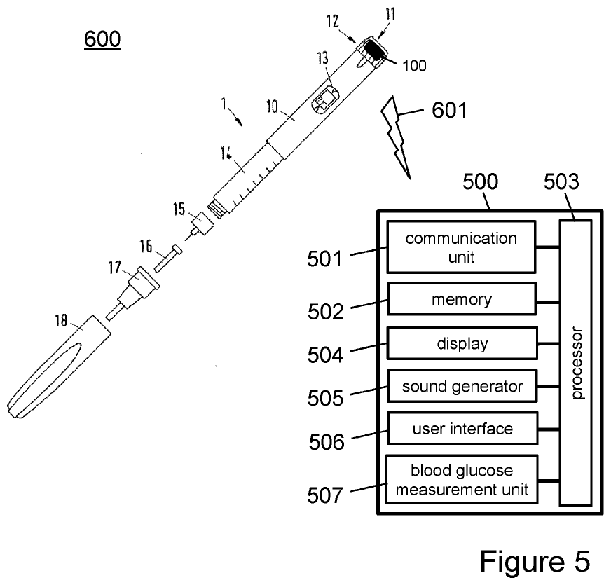

FIG. 5 shows a medical system comprising the drug delivery device of FIG. 1 with the electronic system of FIG. 2 and a second device, wherein the electronic system transmits dose records to the second device, for example in the pattern of FIG. 4.

In the figures, identical elements, identically acting elements, or elements of the same kind may be provided with the same reference numerals.

In the following, some embodiments will be described with reference to an insulin injection device. The present disclosure is, however, not limited to such application and may equally well be deployed with injection devices that are configured to eject other medicaments or drug delivery devices in general, preferably pen-type devices and/or injection devices.

Embodiments are provided in relation to injection devices, in particular to variable dose injection devices, which record and/or track data on doses delivered thereby. These data may include the size of the selected dose and/or the size of the dose actually delivered, the time and date of administration, the duration of the administration and the like. Features described herein include the arrangement of sensing elements and power management techniques (e.g. to facilitate small batteries and/or to enable efficient power usage).

Certain embodiments in this document are illustrated with respect to the injection device disclosed in EP 2 890 435 where an injection button and grip (dose setting member or dose setter) are combined. The injection button may provide the user interface member for initiating and/or performing a dose delivery operation of the drug delivery device. The grip or knob may provide the user interface member for initiating and/or performing a dose setting operation. Both devices are of the dial extension type, i.e. their length increases during dose setting. Other injection devices with the same kinematical behaviour of the dial extension and button during dose setting and dose expelling operational mode are known as, for example, the Kwikpen® device marketed by Eli Lilly and the Novopen® 4 device marketed by Novo Nordisk. An application of the general principles to these devices therefore appears straightforward and further explanations will be omitted. However, the general principles of the present disclosure are not limited to that kinematical behaviour. Certain other embodiments may be conceived for application to the injection device e.g. as described in WO 2004/078239 A1 where there are separate injection button and grip components/dose setting members. Thus, there may be two separate user interface members, one for the dose setting operation and one for the dose delivery operation.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

FIG. 1 is an exploded view of a medicament delivery device or drug delivery device. In this example, the medicament delivery device is an injection device 1, e.g. a pen-type injector, such an injection pen disclosed in EP 2 890 435.

The injection device 1 of FIG. 1 is an injection pen that comprises a housing 10 and contains a container 14, e.g. an insulin container, or a receptacle for such a container. The container may contain a drug. A needle 15 can be affixed to the container or the receptacle. The container may be a cartridge and the receptacle may be a cartridge holder. The needle is protected by an inner needle cap 16 and either an outer needle cap 17 or another cap 18. An insulin dose to be ejected from injection device 1 can be set, programmed, or ‘dialed in’ by turning a dosage knob or dial grip 12, and a currently programmed or set dose is then displayed via dosage window 13, for instance in multiples of units. The indicia displayed in the window may be provided on a number sleeve or dial sleeve. For example, where the injection device 1 is configured to administer human insulin, the dosage may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of number sleeve 301 of a dial sleeve assembly that is configured to move when the dial grip 12 is turned, to provide a visual indication of a currently set dose. The dial grip 12 is rotated on a helical path with respect to the housing 10 when setting a dose.

In this example, the dial grip 12 includes one or more formations to facilitate attachment of a data collection device. Especially, the dial grip 12 may be arranged to attach a button module 11 onto the dial grip 12. As an alternative, the dial grip may comprise such a button module of an electronic system.

The injection device 1 may be configured so that turning the dial grip 12 causes a mechanical click sound to provide acoustic feedback to a user. In this embodiment, the dial grip 12 also acts as an injection button. When needle 15 is stuck into a skin portion of a patient, and then dial grip 12 and/or the attached button module 11 is pushed in an axial direction, the insulin dose displayed in dosage display window 13 will be ejected from injection device 1. When the needle 15 of injection device 1 remains for a certain time in the skin portion after the dial grip 12 is pushed, the dose is injected into the patient's body. Ejection of the insulin dose may also cause a mechanical click sound, which may be different from the sounds produced when rotating the dial grip 12 during dialling of the dose.

In this embodiment, during delivery of the insulin dose, the dial grip 12 is returned to its initial position in an axial movement, without rotation, while the number sleeve 301 is rotated to return to its initial position, e.g. to display a dose of zero units. FIG. 1 shows the injection device 1 in this 0U dialed condition. As noted already, the disclosure is not restricted to insulin but should encompass all drugs in the drug container 14, especially liquid drugs or drug formulations.

Injection device 1 may be used for several injection processes until either the insulin container 14 is empty or the expiration date of the medicament in the injection device 1 (e.g. 28 days after the first use) is reached. In the case of a reusable device, it is possible to replace the insulin container.

Furthermore, before using injection device 1 for the first time, it may be necessary to perform a so-called “prime shot” to remove air from insulin container 14 and needle 15, for instance by selecting two units of insulin and pressing dial grip 12 while holding injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user. Nevertheless, differences (e.g. losses) between the ejected amounts and the injected doses may need to be considered.

As explained above, the dial grip 12 also functions as an injection button so that the same component is used for dialling/setting the dose and dispensing/delivering the dose. As an alternative (not shown), a separate injection button may be used which is axially displaceable, at least a limited distance, relative to the dial grip 12 to effect or trigger dose dispensing.

In the following, an electronic system 100 for a drug delivery device according to the invention will be described with respect to exemplary embodiments.

The electronic system 100 may be implemented in a drug delivery device. In particular, the drug delivery device may include the electronic system 100 shown in FIG. 2. For example, the electronic system 100 may be provided in the injection device 1 of FIG. 1.

In general, the drug delivery device comprises a dose setting and drive mechanism 300 with a first member 20. The drug delivery device further comprises a second member. The first member 20 rotates relative to the second member during the dose delivery operation. The first member 20 may be a dial sleeve assembly or a part thereof. For example, the first member 20 may be a dial sleeve (not shown) of the injection device 1. The dial sleeve may be axially and rotationally fixed to the number sleeve 301. The number sleeve 301 and the dial sleeve may be even formed unitary or be the same component.

The second member may be part of the dose setting and drive mechanism 300 as well. Preferably, the second member is a button module 11, for example as for the injection device 1 shown in FIG. 1. Typically, the button module 11 is mounted to a main part 302 of the dose setting and drive mechanism 300. In more detail, the button module 11 may be axially and rotationally fixed to a member of the dose setting and drive mechanism 300 different from the first member 20, for example to a drive sleeve, a clutch sleeve, or the like. As in the injection device 1 in FIG. 1, the button module 11 may constitute a proximal end of the drug delivery device.

The button module 11 may be in an initial relative position with regard to the first member 20 along the axial direction at least during dose setting operation. The button module 11 is rotationally coupled to the dial sleeve assembly when it is in the initial relative position and hence during dose setting operation. The initial relative position of the button module 11 can be, for example, a most proximal position of the button module 11 relative to the first member 20. Limited axial movement of the button module 11 relative to the first member 20 is allowed. The transition from the dose setting operation to the dose delivery operation includes that the button module 11 is moved, relative to the first member 20, from its initial relative position by at least an axial transition stroke along the axial direction, for example distally. A restoring force may urge the button module 11 towards its initial relative position, for example proximally.

The dose setting and drive mechanism 300 and/or the button module 11 comprise at least one clutch. The clutch is configured such that the transition stroke causes that the dial sleeve assembly and hence the first member 20 (which is the dial sleeve assembly or a part thereof, e.g. the dial sleeve) become rotationally decoupled from the button module 11. This allows that the dial sleeve assembly rotates relative to the button module 11 during dose delivery operation. In other words, the transition stroke and de-coupling of the clutch are necessary for the transition to dose delivery operation and hence for performing dose delivery operation.

The electronic system 100 also comprises a switch 160. In the exemplary embodiments, the switch 160 is a mechanical switch that is operatively connected to the electronic control unit 110.

In one embodiment, the switch 160 includes or consists of an axially activated switch (also referred to as “axial switch”). The switch 160 may be activated by the transition (from dose setting operation) to dose delivery operation, for example by the button module 11 being touched and/or pressed. In more detail, the switch 160 may be an axial switch that is engaged by the axial movement of the button module 11 relative to the first member 20. Preferably, the axial movement of the button module 11 relative due to the first member 20 during the transitions stroke causes the axial switch to engage before the disengagement of the clutch is completed. The electronic control unit 110 can wake up and activate the sensor arrangement 120 before dose delivery operation can actually start.

In this exemplary embodiment, a specific movement is the rotation of the first member 20 relative to the second member, which is the button module 11. The specific movement occurs only during dose delivery operation. As noted above, the dial sleeve assembly (and hence the first member 20) are rotationally de-coupled with regard to the second member (i.e. the button module 11) for dose delivery operation, hence allowing the specific movement to start.

The switch 160 is not limited to an axial switch. It is also not limited to embodiments that are engaged by the transition to dose delivery operation as such. For example, the switch 160 may be a mechanical switch including or consisting of a rotationally activated switch (referred to as rotary switch in the following) that is operatively connected to the electronic control unit 110. The rotary switch may be configured to indicate rotation of the first member 20 relative to the button module 11. The rotary switch is mechanically actuated from said relative rotation. For example, rotation of the first member 20 relative to the button module 11 may cause the rotary switch to switch between a broken circuit state and a closed circuit state thereof. In this case, the switch is engaged directly by (upon) the occurrence of the specific movement. If the rotary switch is engaged, this indicates that dose delivery operation is in progress.

When the switch 160 (axial switch or rotary switch) is engaged, it provides a use signal. If the electronic system 100 is in a sleep state, the electronic control unit 110, more specifically the main microcontroller 111 wakes up upon receiving the use signal, for example by a corresponding interrupt. In response to the use signal, the main microcontroller 111 switches the electronic system 100 immediately to a measurement state. An arrow 201 schematically illustrates a mechanical interaction between the first member 20 and the switch 160.

The button module 11 can comprise the complete electronic system 100 as schematically shown in FIG. 3. The button module 11 is mounted to the main part 302 of the dose setting and drive mechanism 300. In this way, the button module 11 completes the dose setting and drive mechanism 300. The main part 302 includes the first member 20.

The electronic system 100 includes an electronic control unit 110. The electronic control unit 110 may comprise or consist of a PCBA or be part of a PCBA. FIG. 2 schematically shows an exemplary structure of the electronic control unit 110 in more detail. The electronic control unit 110 includes a main microcontroller 111, a memory 112, a sensor controller 113, and a clock generator 114. The main microcontroller 111, the sensor controller 113, the memory 112, and the clock generator 114 are fixed to the PCBA. The electronic control unit 110 is configured to control operation of the electronic system 100.

The memory 112 may comprise or consist of a permanent and/or non-volatile memory. The memory 112 is configured to store data related to the operation of the drug delivery device (for example the injection device 1). In particular, the memory 112 is configured to permanently store dose records described below. The memory 112 may be operatively connected to both the main microcontroller 111 and the sensor controller 113.

The clock generator 114 is configured to provide date and time information. It comprises or consists of a real-time clock. The clock generator 114 may include an oscillator, for example a crystal oscillator. The electronic control unit 110 may comprise further oscillators.

Contacts of the switch 160 may be connected to the electronic control unit 110. The electronic control unit 110 may monitor this electrical connection to determine a circuit state of the mechanical switch 160, for example by means of at least one interrupt.

In other embodiments, in which the first member 20 moves axially relative to the second member during dose delivery operation (not shown), for example distally, the mechanical switch may include or consist of a switch activated by such axial movement.

As shown in FIG. 2, the electronic system 100 further comprises an electrical power supply 150, e.g. a rechargeable or non-rechargeable battery, a communication unit 140, a sensor arrangement 120 for a motion sensor system 129, and optionally an output means 170. The sensor arrangement 120 is operatively connected to the electronic control unit 110. The output means 170 is operatively connected to the electronic control unit 110 as well.

The communication unit 140 is configured for communicating with a second device 500. The communication unit 140 is included in or operatively connected to the electronic control unit 110. In order to save electrical energy, the communication unit 140 may be active only in a pairing state for pairing with the second device 500 for wireless communications and in a synchronization state. Typically, the electronic system 110 switches the electronic system 100 automatically to the synchronization state after dose delivery operation has been finished. In the synchronization state, the communication unit 140 transmits dose records to the second device 500 that have not been transmitted yet. The electronic system 110 may be allowed to manually activate the paring state and/or the pairing state.

The output means 170 may comprise a LED indicator, a sound generator, a vibration alert means, and/or a display.

Unless specifically disclosed otherwise, the electronic system 100 may have the functions and may be arranged and/or designed as described in WO 2019/101962 A1, unpublished EP 20315357.2, EP 20315066.9, EP 20315451.3, and EP 21315002.2, the disclosure of which is incorporated herein by reference.

FIG. 3 schematically shows the combination of the electronic system 100 of FIG. 2 with the first member 20 of the dose setting and delivery mechanism 300. As noted above, the first member 20 may be the dial sleeve assembly of the dose setting and delivery mechanism 300 or a part of the dial sleeve assembly, for example the dial sleeve.

Power consumption of the sensor arrangement 120 and hence of the electronic system 100 may be particularly high while the sensor controller 113 operates the sensor arrangement 120. The power management with regard to the sensor arrangement 120 may have particular impact on the lifetime of the battery used as electrical power supply 150.

Preferably, the electronic control unit 110 is configured such that (only) the sensor controller 113 operates the sensor arrangement 120 in the measurement state. The electronic control unit 110 may be configured such that the sensor controller 113 is switched off (not operated) in any state of the electronic system 100 different from the measurement state.

The motion sensor system 129 comprises the sensor arrangement 120 and an encoder component 125 (see FIG. 3). In this embodiment, the motion sensor system 129 is a rotary sensor system and the encoder component 125 is axially and rotationally coupled to the first member 20. The encoder component 125 may be formed integrally with the first member 20.

The sensor arrangement 120 is operable by the electronic control unit 110 to generate (provide) sensor data describing the specific movement of the first member 20 relative to the second member (i.e. the button module 11). An overall specific movement during dose delivery operation corresponds to a size of the dose delivered during the respective dose delivery operation. For example, the size of the dose delivered is proportional to an overall extent of rotation of the first member 20 with regard to the button module 11 during the complete dose delivery operation. Especially if the size of the dose is large, the extent of rotation may comprise several complete revolutions (each) 360° of the first member 20 relative to the button module 11.

The motion sensor system 129 and especially the sensor arrangement 120 may be implemented in accordance with any one of the embodiments disclosed in WO 2019/101962 A1 and EP 20315066.9 (which are incorporated by reference), for example. Preferably, the motion sensor system 129 and especially the sensor arrangement 120 is/are implemented according to any one of the embodiments disclosed in EP 20315357.2, which is also incorporated by reference.

The sensor arrangement 120 comprises one sensor or a plurality of sensors 122a, 122b. In the embodiment shown in FIG. 2, the two sensors 122a, 122b are optoelectronic sensors for detecting electromagnetic radiation, such as IR sensors. The sensors 122a, 122b may be angularly separated (in particular along a circumferential direction around an axis of relative rotation between the first member 20 and the button module 11). The sensor arrangement 120 may additionally comprise at least one radiation emitter 121a, 121b which emits radiation to be detected. Each sensor 122a, 122b may have an associated radiation emitter 121a, 121a as in FIG. 2. The encoder component 125 may comprise a plurality of angularly separated detection regions. The detection regions may have a higher reflectance for the emitted radiation than regions in-between adjacent detection regions (non-detection regions).

In FIG. 3, an arrow 202 schematically illustrates an interaction between the sensor arrangement 120 and the encoder component 125. In this embodiment, the encoder component 125 is an encoder ring that is axially and rotationally fixed to the first member 20 (for example the dial sleeve). The radiation emitters 121a, 121b emit light (which also may mean IR light and/or UV light) and the light can be reflected by the angularly separated detection regions of the encoder component 125. Depending on the relative rotational position between the first member 20 and the second member (button module 11), the detection regions face different sensors 122a, 122b. A detection pattern, which of the sensors 122a, 122b detects high reflection, depends on and changes with said relative rotational positions. It depends on said relative rotational position whether both sensors 122a, 122b, only the second sensor 122b, only the first sensor 122a, or none of the sensors 122a, 122b detects a high reflection of radiation. Hence, sensor data from the sensor arrangement 120 allows to distinguish between four different successive (rotational) positions of the first member 20 relative to the button module 11.

In the exemplary embodiment, the sensor arrangement 120 is configured to produce or form a Gray code (at least when operated in combination with the encoder component 125). Particularly, data on two adjacent positions differ in only one bit. Changes (transitions) of the code output indicate that the first member 20 moves relative to the second member (i.e. the button module 11).

When the first member 20 rotates during dose delivery operation relative to the button module 11, for example anti-clockwise, the two sensors 122a and 122b produce 2-bit Gray code outputs (11, 01, 00, 10). The 2-bit code sequence repeats every four units dispensed.

The first bit is ‘1’ if the first sensor 122a faces any one of the detection regions of the encoder component 125 and is ‘0’ else (i.e. when the first sensor 122a faces any one of the non-detection regions of the encoder component 125). The second bit is ‘1’ if the second sensor 122b faces any one of the detection and is ‘0’ else (i.e. when the second sensor 122b faces any one of the non-detection regions). As an example, the four possible code outputs may be simply indicated by values 0, 1, 2, 3. During relative rotation, the code output (and hence the corresponding Gray code values) may repeat after each one-sixth revolution, for example.

As an example, the sensor arrangement 12 may provide the same Gray code value 0 when the rotational position of the first member 20 relative to the button module 11 is in the following relative angular position ranges: 1° to 15°, 61° to 75°, 121° to 135°, 181° to 195°. 241° to 255° and 301° to 315; and the Gray code value 1 is provided when the rotational position of the first member 20 relative to the button module 11 is in the following relative angular position ranges: 16° to 30°, 76° to 90°, 136° to 150°, 196° to 210°, 256° to 270°, and 316° to 330°. Accordingly the Gray code value 2 is provided for six other relative angular position ranges and the Gray code value 3 is provided for further six other relative angular position ranges.

Naturally, a Gray code resolution can be enhanced easily if more than two sensors 122a, 122b are used. This allows to distinguish between more different subsequent positions of the first member 20 relative to the button module 11.

However, other motion sensor systems can be employed as well. For example, the motion sensor system 129 may additionally or alternatively comprise a magnetic rotational sensor system, a mechanical rotational sensor system and/or an inductive rotational sensor system.

This code output facilitates the detection of “forward” specific movement (anticlockwise rotation) and “backward” specific movement (clockwise rotation). Forward specific movement indicates that the size of the dose being dispensed at the moment is increasing. For example, when the sensor arrangement reads ‘11’, a change (transition) to ‘01’ would be a forward rotation and the change to ‘10’ would be a backward rotation. This directionally sensitive system has advantages over a purely incremental system, in the ability to accurately determine true dispensed dose volume in the cases where backward rotations can occur. For example, the first member 20 of the dose setting and drive mechanism 300 may tend to over-rotate at the end of dose delivery operation before ‘backing-off’ when the user releases the button module 11. However, preferably forward specific movement should occur during dose delivery operation. The electronic system 100 may be configured to consider only forward specific movement for determining the size of the dose delivered, for checking whether any one of the speed thresholds is breached, and/or for calculating a speed of the specific movement (and/or a dispense speed corresponding to the specific movement).

In the measurement state, the electronic control unit 110 operates the sensor arrangement 120 periodically with a lower sampling rate (“base” sampling rate) and switches to operate the sensor arrangement with a second, high sample rate (“fast” sample rate) when the code output indicates any transition. For example, the fast sample rate may be at least 3500 Hz, for example 4000 Hz. The base sample rate may be at least 100 Hz but less than the fast sample rate, for example 500 Hz. Furthermore, the electronic control unit 110 switches back to operate the sensor arrangement 120 with the (“base” sample rate) after having operated the sensor arrangement 120 with the fast sample rate for a pre-defined period of time and/or after having obtained a pre-defined number of sensor readings (code outputs) with the first fast sample rate. Counting the pre-defined period of time and/or the pre-defined number of sensor readings may be reset when the code output indicates any new transition. This prevents the sample rate from being reduced although transitions continue to occur frequently.

The electronic control unit 110, for example specifically the sensor controller 113, is configured to determine the size of the dose delivered during dose delivery operation. For example, the electronic control unit 110 may calculate the size of the dose based on the sensor data obtained from the sensor arrangement 120 during dose delivery operation in the measurement state.

A change rate of the code outputs (transition(s) per time unit or time per transition(s)) corresponds to the speed of the specific movement and hence to the dispense speed. If the change rate is considered in terms of transition(s) per time unit, the change rate of the code output linearly corresponds to the specific movement and the speed of the dose delivery operation. If the change rate is considered in terms of time per transition(s), the change rate inversely corresponds to the speed of the specific movement and hence to the dispense speed. The dose setting and drive mechanism 300 transforms the rotation of the dose dial assembly (with the first member 20) relative to the second member and the housing 10 to an axial movement of a piston rod in the distal direction. Hence, the piston rod can urge a bung in the container 14 in distally (i.e. forward) for expelling fluid from the container 14.

Especially, the time interval between two consecutive transitions corresponds to the instantaneous speed of the specific movement (and hence the dispense speed). By considering the time needed for a certain number of transitions (i.e. a time interval from a first transition to a last transition of the certain number of transitions), the speed detection (or measurement) can be averaged. Similarly, counting the number of transitions within a pre-defined time period can allow the checking of whether specific speed thresholds are breached and/or to calculate the speed.

In the exemplary embodiment, in the context of normal dose delivery operation, the following sequence of events may occur and the electronic system 100 and the injection device 1 are adapted accordingly:
During dose setting operation, the user rotates the dial sleeve assembly to set the desired dose. The dial sleeve assembly may helically wind out of the housing 10 when the set dose is being increased.
The button module 11 is depressed by at least the transition stroke. The button module 11 is rotationally decoupled from the dial sleeve assembly with the first member 20. The dial sleeve assembly with the first member 20 starts to rotate relative to the button module 11 for the dose delivery operation. The switch 160 generates a use signal upon pressing the button module 11 and/or upon the rotation of the first member 20 relative to the button module 11.
The electronic control unit 110 switches the electronic system 100 to the measurement state based on the use signal. In more detail, the main microcontroller 111 configures and starts the sensor controller 113 for operating the sensor arrangement 120 in the measurement state.
In the measurement state, the sensor controller 113 operates the sensor arrangement 120, for example the radiation emitter 121A, 121B and the optical sensors 122A, 122B thereof, to provide sensor data describing rotation of the first member 20 relative to the button module 11.
The dose is dispensed.
The electronic control unit 110 determines the size of the dose based at least on the sensor data obtained in the measurement state.
When dispense is completed, the electronic control unit 110 operates the clock generator 114 to obtain date and time information and stores a dose record 400 for this dose delivery operation.
The electronic system 110 may automatically switch the electronic system 100 to another state, for example to the synchronization state, after dose delivery operation has been finished and if a new dose record is available.
In the synchronization state, the communication unit 140 transmits the new dose record to the second device 500. If previous dose records have not been transmitted yet to the second device 500, they are transmitted to the second device 500 as well.

A dose record pattern 400 for the dose record includes at least a time stamp field 401 and a dose size field 402. The time stamp field 401 is suitable to store the date and time information provided by the clock generator 114. The dose size field 402 is suitable to store the size of the dose.

The electronic control unit 110 checks whether the dispense speed breaches a first speed threshold. In the exemplary embodiment, the first speed threshold is an upper threshold, in more detail a very fast speed threshold for dose delivery operation. If the very fast speed threshold is exceeded, this indicates that an applicable measurement capability of the electronic system 100 has been almost reached. The applicable measurement capability may be inherent to the motion sensor system 129 due to the hardware or may be given be a pre-programmed maximum sample rate, for example the first sample rate. The pre-programmed maximum sample rate may be less than a highest sample rate that would be possible with the hardware in order to reduce the electrical power consumption.

In the exemplary embodiment, the very fast speed threshold is defined as a single transition time threshold of 1.0 ms±0.5 ms. If the time interval between two subsequent increasing transitions during dose delivery operation is shorter than this, the speed of the specific movement during (the at least one part of) the dosing operation exceeds the very fast speed threshold.

Preferably, the electronic control unit 110 raises a “very fast speed flag” in the dose record if the dispense speed during the respective dose delivery operation has exceeded the very fast speed threshold, for example by setting a value in a “very fast speed flag field” 403 in the dose record to a value corresponding to ‘true’ in this case and to a value corresponding to ‘false’ else (i.e. the “very fast speed flag” is not raised else). Additionally or alternatively, the electronic control unit 110 may set the time stamp field 401 and/or the dose size field 402 (preferably only the dose size field 402) to a specific purpose value indicating that the dispense speed during the respective dose delivery operation has exceeded the very fast speed threshold.

In one embodiment, the electronic control unit 110 operates the output means 170 to (immediately) provide a very fast speed warning to the user. For example, the LED indicator may indicate a visual very fast speed warning, the display may indicate a graphic and/or text-based very fast speed warning, and/or a sound generator may generate an acoustic fast speed warning. The very fast speed warning may include further information, for example the calculated speed, a maximum applicable speed (corresponding to the applicable measurement capability), and/or a specific indication that the maximum speed is almost reached.

The electronic control unit 110 checks whether the dispense speed breaches a second speed threshold. In the exemplary embodiment, the second speed threshold is another upper threshold, in more detail a fast speed threshold for dose delivery operation. The fast speed threshold corresponds to a slower speed than the very fast speed threshold.

In the exemplary embodiment, the fast speed threshold is a multiple transitions time threshold and defines a minimum acceptable time interval of 140 ms±4 ms for 8 consecutive increasing transitions. If the overall time interval needed for 8 consecutive increasing transitions during dose delivery operation is shorter, the electronic control unit 110 determines that the speed of dose delivery operation exceeds the fast speed threshold.

Additionally or alternatively, the fast speed threshold may be defined to correspond to a certain dispense speed, wherein the certain dispense speed is in the range from 20 units/s to 150 units/s, preferably from 30 units/s to 100 units/s, for example 50 units/s. If the sensor data indicates a higher dispense speed, the electronic control unit 110 determines that the speed of dose delivery operation exceeds the fast speed threshold.

Preferably, the electronic control unit 110 is configured to raise a “fast speed flag” in the dose record if the dispense speed has exceeded the fast speed threshold, for example by setting a value in a “fast speed flag field” 404 in the dose record to a value corresponding to ‘true’ in this case and to a value corresponding to ‘false’ else (i.e. the “fast speed flag” is not raised else). Additionally or alternatively, the electronic control unit 110 is configured to set the time stamp field 401 and/or the dose size field 402 (preferably only the dose size field 402) to a specific purpose value indicating that the dispense speed has exceeded the fast speed threshold.

In one embodiment, the electronic control unit 110 operates the output means 170 to (immediately) provide a fast speed warning to the user. For example, the LED indicator may indicate a visual fast speed warning, the display may indicate a graphic and/or text-based fast speed warning, and/or the sound generator generates an acoustic fast speed warning. The fast speed warning may indicate that the dispense speed is unfavourably high. The fast speed warning may include additional information, for example that a fast dispense speed may increase the risk of a painful injection, a recommended dispense speed, and/or the calculated dispense speed. Furthermore, the fast speed warning may include information that a fast dispense speed can indicate that no cartridge is fitted, air is dispensed, and/or the needle is not penetrating the patient's skin. Additionally or alternatively, the fast speed warning may include an invitation to check for these issues. Naturally, the very fast speed warning can include parts of or all of this information as well because exceeding the very fast speed threshold includes exceeding the fast speed threshold.

Immediately providing the corresponding very fast speed warning and the fast speed warning under the respective conditions ensures that the user immediately is made aware of the problem. The user receives direct feedback with regard to their operation of the injection device 1. This facilitates the user to alter their behaviour and to apply more favourable dispense speeds.

Providing the very fast speed indication (i.e. raising the very fast speed flag) and the very fast speed indication (i.e. raising the very fast speed flag) in the dose record in case of the respective conditions allows the following up later whether the speed thresholds were breached during the respective dose delivery operation. This helps to recognize, monitor, and/or improve the injection behaviours of patients, health care professionals (HCP), and other users.

Furthermore, the speed indications stored in the dose record may help the patient, the user, the HCP, the manufacturer, and/or the second device 500 to decide whether the corresponding individual dose record should be excluded from further evaluation or should be considered only with a reduced weight.

According to a further aspect, the electronic system 100 may be configured to set a value of the calculated dispense speed in a dispense speed field 405 in the dose record. It is possible that the dispense speed field 405 is only included in the data record pattern 400 of the dose record if at least one of the speed thresholds was breached. Alternatively, the dispense speed field 405 is included in any dose record. In this case, the very fast speed flag field 403 and/or the fast speed flag field 404 could be omitted. The second device 500 may determine on its own whether any speed thresholds have been breached based on the calculated speed stored in the dispense speed field 405.

FIG. 5 shows a medical system 600 comprising the injection device 1 illustrated in FIG. 1 including the electronic system 100 (shown in FIG. 2 and FIG. 3) and the second device 500. The electronic system 100 is incorporated in the button module 11 of the injection device 1. In the embodiment, the second device 500 is a blood glucose meter. FIG. 5 schematically illustrates wireless transmission 601 of the dose records from the electronic system 100 to the second device 500.

The second device 500 comprises a communication unit 501 for receiving the dose records transmitted from the electronic system 100, a memory 502 configured to store the received dose records, a processor 503, and a display 504. The second device 500 may be further adapted to receive measurement values for a body property of the patient, e.g. blood glucose measurement values, for example by the receiving means 501 and/or a user interface 505. The user interface 505 comprises, for example, a touchscreen functionality of the display 504, a memory card slot, a keyboard, a mouse, a voice command unit, and/or a gesture command unit. The processor 503 is configured to control operation of the second device 500.

The second device 500 may be configured to
analyse the dose records, to detect risks for the patient based on the dose records,
to provide warnings and/or recommendations based on the dose records, for example by means of the display and/or a sound generator 506, and/or
to determine a dose of medicament (e.g. insulin) to be set based on the dose records and the measurement values for the body property.

In particular, the second device 500 may include a dose helper functionality. The dose helper functionality may include at least one titration method for stepwise adapting doses of insulin to be set based on the dose records and the blood glucose measurement values. The processor 503 is configured to execute the titration method. In this embodiment, the second device 500 itself includes a blood glucose measurement unit 507 for providing blood glucose measurement values.

Preferably, the second device 500 is adapted to check whether the respective individual dose record indicates that any of the speed thresholds has been breached. If the dose record includes the calculated speed, the second device 500 may check itself whether the calculated speed breaches the very fast speed threshold, the fast speed threshold, and/or at least one further speed threshold. Additionally or alternatively, the second device 500 can simply check whether any speed indications (like the very fast speed flag and/or the fast speed flag) are included in the dose record.

The second device 500 can be configured to provide speed warnings. The explanations regarding the speed warnings of the electronic system 100 may apply accordingly. Hence, the second device 500 can make e.g. the HCP aware of unfavourable dispense behaviour of the user. The HCP then can instruct the user to change their behaviour.

The second device 500 may be also configured to exclude dose records from further evaluation or change the weight for evaluation, for example with the dose helper functionality, depending on which speed thresholds were breached. For example, at least dose records indicating that the very fast speed threshold was exceeded during the respective dose delivery operation may be excluded from further evaluation.

In another embodiment, the second device is a smartphone (not shown). The smartphone may include the dose helper functionality.

By monitoring the dispense event and calculating and analysing the dispense speed, useful information can be provided to the patient, the user, the HCP, the manufacturer and/or the medical device, especially useful information in relation to the behaviour of the user operating the drug delivery device. The information can subsequently be used to train the user to improve or alter the behaviour.

The present invention allows to detect inappropriate dosing speeds and to warn users before dose record errors occur. Information is provided how fast the users are dosing. The information can be provided to the patient, the user, the HCP, the manufacturer, and/or the second device 500. In particular, speeds of the dosing operation close to, at, and/or beyond the measurement capability of the electronic system can be detected. Accordingly, the users can easily learn to avoid inappropriate speeds of the dosing operation. This helps to increase the reliability and accuracy of data provided from the electronic system (for example of the dose records) and also helps to avoid particularly painful injections.

### REFERENCE NUMERALS

1 injection device (drug delivery device)
10 housing
11 button module (second member)
12 dial grip
13 dosage window
14 container/container receptacle
15 needle
16 inner needle cap
17 outer needle cap
18 cap
20 first member
100 electronic system
110 electronic control unit
111 main microcontroller
112 memory
113 sensor controller
114 clock generator
120 sensor arrangement
121a, 121b radiation emitter
122a, 122b sensor
125 encoder component
129 rotary sensor system
140 communication unit
150 electric power supply
160 switch
170 output means
201, 202 arrow
300 dose setting and drive mechanism
301 number sleeve
302 main part (of the dose setting and drive mechanism)
400 data record pattern
401 time stamp field
402 dose size field
403 very fast speed flag field
404 fast speed flag field
405 dispense speed field
500 second device
501 communication unit
502 memory
503 processor
504 display
505 interface
506 sound generator
507 blood glucose measurement unit
600 medical system
601 transmission

1. An electronic system (100) for a drug delivery device (1), in which a first member (20) performs a specific movement relative to a second member (11) during a dosing operation,
wherein the electronic system (100) comprises a sensor arrangement (120), which is operable to provide sensor data describing the specific movement, and wherein the electronic system (100) is configured to operate the sensor arrangement (120) during at least one part of the dosing operation to provide sensor data;
characterized in that the electronic system (100) is configured to determine, based on the sensor data,
if a speed of the specific movement during the at least one part of the dosing operation breaches a first speed threshold, and/or
the speed of the specific movement and/or the dosing operation during the at least one part of the dosing operation.

2. The electronic system (100) according to claim 1, wherein the electronic system (100) is configured to determine, based on the sensor data, if the speed of the specific movement during the at least one part of the dosing operation breaches the first speed threshold, wherein the first speed threshold is a very fast speed threshold which is set to a value which is sufficiently high that a user will not exceed the corresponding speed in normal dosing operation.

3. The electronic system (100) according to claim 1, wherein the electronic system (100) is configured to determine, based on the sensor data, if the speed of the specific movement during the at least one part of the dosing operation breaches the first speed threshold, wherein the electronic system (100) is configured to operate the sensor arrangement (120) periodically with a first sample rate during the at least one part of the dosing operation, wherein the first speed threshold is a very fast speed threshold, which corresponds to
a maximum time resolution of the sensor arrangement (120) and/or the first sample rate or
to a certain proportion to the maximum time resolution and/or the first sample rate.

4. The electronic system (100) according to any one of the preceding claims, wherein the electronic system (100) is configured to determine, based on the sensor data, if the speed of the specific movement during the at least one part of the dosing operation breaches the first speed threshold, wherein the electronic system (100) comprises at least one further speed threshold and is configured to determine, based on the sensor data, if the speed of the specific movement during the at least one part of the dosing operation breaches the at least one further speed threshold, wherein the at least one further speed threshold is less than the first speed threshold.

5. The electronic system (100) according to any one of the preceding claims, wherein the electronic system (100) is configured to provide a first speed warning depending on whether the specific movement during the at least one part of the dosing operation breaches the first speed threshold.

6. The electronic system (100) according to any one of the preceding claims, wherein the electronic system (100) comprises a memory (112) and/or a communication unit (140) for the transmission (601) of data to a second device (500),
wherein the electronic system (100) is configured to generate a data record for the dosing operation and to include a first speed indication in the data record depending on whether the specific movement during the at least one part of the respective dosing operation breaches the first speed threshold, and
wherein the electronic system (100) is configured to store the data record in the memory (112) and/or to transmit the data record to the second device (500).

7. The electronic system (100) according to any one of the preceding claims, wherein the electronic system (100) is configured to operate the sensor arrangement (120) at least with the first sample rate and a second sample rate, wherein the first sample rate is higher than the second sample rate, and
wherein the electronic system (100) is configured to operate the sensor arrangement (120) with the second sample rate and to increase the sample rate to the first sample rate when the sensor data indicates occurrence of the specific movement.

8. The electronic system (100) according to claim 7, wherein the electronic system (100) is configured to reduce the sample rate below the first sample rate, for example to the second sampling rate,
after having operated the sensor arrangement (120) continuously with the first sample rate for a pre-defined time and/or
having obtained a pre-defined number of sensor readings in one turn with the first sample rate.

9. The electronic system (100) according to any one of the preceding claims, wherein the electronic system (100) comprises a switch (160) configured to be engaged by
by a transition to dose delivery operation, and/or
by the occurrence of the specific movement,
wherein the electronic system (100) is configured to switch to a measurement state when the mechanical switch (160) is engaged.

10. The electronic system (100) according to claim 9, wherein the electronic system (100) comprises a main microcontroller (111) and a sensor controller (113), wherein the electronic system (100) is configured such that the sensor controller (113) operates the sensor arrangement (120) in the measurement state and that the sensor controller (113) is in a power-saving condition else.

11. The electronic system (100) according to any one of the preceding claims, wherein the sensor arrangement (120) is operable to provide sensor data indicating transitions between subsequent positions of the first member (20) relative to the second member (11), wherein the electronic system (100) is configured to determine that the speed of the specific movement during the at least one part of the dosing operation exceeds the first speed threshold if the sensor data indicates that
a time between consecutive transitions is below a single transition threshold value,
a time between a certain number of transitions is below a multiple transition threshold value, and/or
a number of transitions in a given threshold time is greater than a certain value.

12. The electronic system (100) according to any one of the preceding claims, wherein the electronic system (100) is configured to determine the speed of the specific movement during the at least one part of the dosing operation based at least on the sensor data.

13. The electronic system (100) according to any one of the preceding claims, wherein the dosing operation includes
a dose delivery operation for delivering a dose by the drug delivery device (1), wherein a size of the dose to be delivered is user-settable or pre-defined, and/or
a dose setting operation for setting the size of the dose to be delivered by the drug delivery device (1).

14. The electronic system (100) according to claim 11 or 12, wherein the overall specific movement of the first member (20) relative to the second member (11) during dosing operation corresponds to the size of the dose, wherein the electronic system (100) is configured to determine the size of the dose based at least on the sensor data obtained by operating the sensor arrangement (120) during the dosing operation.

15. A drug delivery device (1) comprising the electronic system (100) according to any one of the claims 1 to 13, wherein the drug delivery device (1) further comprises a container receptacle which is adapted to receive a container (14) containing a medicament.

16. Method for operating an electronic system (100) for a drug delivery device (1), preferably the electronic system (100) according to any one of the preceding claims 1 to 13 or the electronic system (100) of the drug delivery device (1) according to claim 14,
wherein a first member (20) of the drug delivery device (1) performs a specific movement relative to a second member (11) of the drug delivery device (1) during a dosing operation,
wherein the electronic system (100) comprises a sensor arrangement (120), which is operable to provide sensor data describing the specific movement, wherein the electronic system (100) operates the sensor arrangement (120) during at least one part of the dosing operation to provide sensor data;
characterized in that the electronic system (100) determines, based on the sensor data,
if a speed of the specific movement during the at least one part of the dosing operation breaches a first speed threshold, and/or
the speed of the specific movement and/or the dosing operation during the at least one part of the dosing operation.
